SEQUENCE LISTING (5 sequences)

SEQ ID NO: 1 - DNA, 3017 bp, Homo sapiens
ttcagagaaatccctgaattcactgaaagttttatctagaaatacatgtgcaagtgaacacatcttttttaaaaaaaatcattacctactttcttttttgagaagaaggtatttatttcaacagactcttgaaggagcctactcttcccactctcccacccccattaagaaccactgtaggccgggcacgatggctcatgcctgtaatcccagcactttgggaggctaaggtgggtggatcacctgaggtcaggagttcgagacaagcctagccaacatagtgaaaccccgtctctactaataatacaaaaattagctgggtatggcagcatgtgcctgtaatcccagctactcgggaggctgaggcaggagaattgctcgaacccgggaggcggaggttgcagtgaaccgagagagatcgtgcggtgccatttcactccagcctgggcaacagagcgaaactccatctcaaaaaaacacacaaaacaaacaaacaaaaagaaagaaccattgtattagtgatggaaatgtgttccctccctcccatcctggcaaccactttcttcctcctccatcataaaatatcttaaactaaactaaaataattttatttatcgatagtttgaattttccctatcattgctacacagctaattgagaggtaccccgaggaaaatataaatggtacagtaatgcattgtagattttaataacatacttgacatcccaaattgttttcattggcttcattttaaaaactacatgttttaaaatcaagcagacactaaaagtacaagatatactgggtctacaaggtttaagtcaaccagggattgaaatataacttttaaacagagctggattatccagtaggcagattaagcatgtgcttaaggcatcagcaaagtctgagcaatccattttttaaaacgtagtacatgtttttgataagcttaaaaagtagtagtcacaggaaaaattagaacttttacctccttgcgcttgttatactctttagtgctgtttaacttttctttgtaagtgagggtggtggagggtgcccataatcttttcagggagtaagttcttcttggtctttctttctttctttctttctttttttcttgagaccaagtttcgctcttgtctcccaggctggagtgcaatggcgcgatctcggctcactgcaacctccgccttctcctgggttcaagcgattctcctacatcagcctccgagtagctgggattacaggcatgcgccaccaagccccgctaattttgtattttttagtagagacagggtttcgccatgttggtcaggcttgtctcgaactcctggcctcaggtgatccgcctgtctcggcctcccagaatgctgggattatagacgtgagccaccgcatccggactttccttttatgtaatagtgataattctatccaaagcatttttttttttttttttgagtcggagtctcattctgtcacccaggctggagggtggtggcgcgatctcggcttactgcaacctctgcctcccgggttcaagcgattctcctgcctcagcctcctgagtagctggaattacacacgtgcgccaccatggccagctaatttttgtatttttagtagagacggggtgtcaccattttggccaagctggcctcgaactcctgacctcaggtgatctgcccgcctcggcttcccaaagtgctgggattacaggtgtgagccaccgcgtcctgctccaaagcattttctttctatgcctcaaaacaagattgcaagccagtcctcaaagcggataattcaagagctaacaggtattagcttaggatgtgtggcactgttcttaaggcttatatgtattaatacatcatttaaactcacaacaacccctataaagcagggggcactcatattcccttccccctttataattacgaaaaatgcaaggtattttcagtaggaaagagaaatgtgagaagtgtgaaggagacaggacagtatttgaagctggtctttggatcactgtgcaactctgcttctagaacactgagcactttttctggtctaggaattatgactttgagaatggagtccgtccttccaatgactccctccccattttcctatctgcctacaggcagaattctcccccgtccgtattaaataaacctcatcttttcagagtctgctcttataccaggcaatgtacacgtctgagaaacccttgccccagacagccgttttacacgcaggaggggaaggggaggggaaggagagagcagtccgactctccaaaaggaatcctttgaactagggtttctgacttagtgaaccccgcgctcctgaaaatcaagggttgagggggtagggggacactttctagtcgtacaggtgatttcgattctcggtggggctctcacaactaggaaagaatagttttgctttttcttatgattaaaagaagaagccatactttccctatgacaccaaacaccccgattcaatttggcagttaggaaggttgtatcgcggaggaaggaaacggggcgggggcggatttctttttaacagagtgaacgcactcaaacacgcctttgctggcaggcgggggagcgcggctgggagcagggaggccggagggcggtgtggggggcaggtggggaggagcccagtcctccttccttgccaacgctggctctggcgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagc

SEQ ID NO: 2 - DNA, 20 bp, homo sapiens
gggcatgtccgggcatgtcc

SEQ ID NO: 3 - DNA, 1251 bp, HOMO SAPIENS
atggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataa

SEQ ID NO: 4 - DNA, 834 bp, HOMO SAPIENS
atggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaa

SEQ ID NO: 5 - DNA, 1089 bp, HOMO SAPIENS
atgctccagaagcccaagagcgtgaagctgcgggccctgcgcagcccgaggaagttcggcgtggctggccggagctgccaggaggtgctgcgcaagggctgtctccgcttccagctccctgagcgcggttcccggctgtgcctgtacgaggatggcacggagctgacggaagattacttccccagtgttcccgacaacgccgagctggtgctgctcaccttgggccaggcctggcagggctgtgagtggcaaggactttggaggatgtgtcttctgctggaccggcaccttttgtttgtcccattggtggcagatgtgagcgacatcaggcgcttcctcagtgcatttcacgagccacaggtggggctcatccaggccgcccagcagctgctgtgtgatgagcaggccccacagaggcagaggctgctggctgacctcctgcacaacgtcagccagaacatcgcggccgagacccgggctgaggacccgccgtggtttgaaggcttggagtcccgatttcagagcaagtctggctatctgagatacagctgtgagagccggatccggagttacctgagggaggtgagctcctacccctccacggtgggtgcggaggctcaggaggaattcctgcgggtcctcggctccatgtgccagaggctccggtccatgcagtacaatggcagctacttcgacagaggagccaagggcggcagccgcctctgcacaccggaaggctggttctcctgccagggtccctttgacatggacagctgcttatcaagacactccatcaacccctacagtaacagggagagcaggatcctcttcagcacctggaacctggatcacataatagaaaagaaacgcaccatcattcctacactggtggaagcaattaaggaacaagatggaagagaagtggactgggagtatttttatggcctgctttttacctcagagaacctaaaactagtgcacattgtctgccataagaaaaccacccacaagctcaactgtgacccaagcagaatctacaaaccccagacaaggttgaagcggaagcagcctgtgcggaaacgccagtga

(19) United States
(12) Patent Application Publication
(10) Pub. No.: US 2016/0010110 A1
(43) Pub. Date: Jan. 14, 2016

(54) SYSTEMS AND METHODS FOR THE TARGETED PRODUCTION OF A THERAPEUTIC PROTEIN WITHIN A TARGET CELL

(71) Applicant: OISIN BIOTECHNOLOGIES, Seattle, WA (US)

(72) Inventor: Matthew R. Scholz, Seattle, WA (US)

(73) Assignee: OISIN BIOTECHNOLOGIES, Seattle, WA (US)

(21) Appl. No.: 14/862,161

(22) Filed: Sep. 23, 2015

Related U.S. Application Data
(63) Continuation of application No. PCT/US14/31638, filed on Mar. 24, 2014.
(60) Provisional application No. 61/804,716, filed on Mar. 24, 2013.

(51) Int. Cl. C12N 15/85

(52) U.S. Cl. CPC C12N 15/85; A61K 48/00

(57) ABSTRACT

Provided are nucleic acid-based expression constructs for the targeted production of a therapeutic protein within a cell that is associated with aging, disease, another condition. Also provided are vectors and systems for the delivery of those nucleic acid-based expression constructs as well as methods for using such nucleic acid-based expression constructs, vectors, and systems for reducing, preventing, and/or eliminating the growth and/or survival of an age-, disease-, or condition-associated cell and for the treatment of a disease or condition that is associated with an age, disease, or condition associated cell.

### CROSS-REFERENCE TO RELATED APPLICATIONS

This U.S. non-provisional patent application was filed on Sep. 23, 2015 and claims priority as a continuation to PCT Patent Application No. PCT/US2014/031638, which was filed on Mar. 24, 2014 and claims the benefit of U.S. Provisional Patent Application No. 61/804,716, which was filed on Mar. 24, 2013. The contents of PCT Patent Application No. PCT/US2014/031638 and U.S. Provisional Patent Application No. 61/804,716 are incorporated herein by reference in their entirety.

### BACKGROUND OF THE DISCLOSURE

1. Technical Field

The present disclosure relates, generally, to the field of medicine, including the treatment of disease, promotion of longevity, anti-aging, and health extension. More specifically, this disclosure concerns compositions and methods for reducing the growth and/or survival of cells that are associated with aging, disease, and other conditions. Provided are expression constructs for target cell specific expression of therapeutic proteins, which constructs exploit unique intracellular functionality, including transcription regulatory functionality, that is present within a target cell but is either absent from or substantially reduced in a normal, non-target cell. Such expression constructs are used in systems that include a vector for the delivery of a nucleic acid to a target cell, which vectors may comprise, but do not necessarily require, a targeting moiety for enhancing the delivery of an expression construct to a target cell.

2. Description of the Related Art

Cancer cells, senescent cells, and other cells having an undesirable phenotype can accumulate over the course of a person's life and, without appropriate treatment, such cells can contribute to or even cause a person's morbidity and, ultimately, mortality.

The role of senescent cells in disease and the potential benefits of eliminating senescent cells has been discussed in scientific publications such as Baker et al. Nature 479:232-6 (2011). Systems and methods have been described that purport to address the problem of accumulating senescent cells. For example, Grigg, PCT Patent Publication No. WO 1992/009298, describes a system for preventing or reversing cell senescence with chemical compounds similar to carnosine and Gruber, U.S. Patent Publication No. 2012/0183534, describes systems for killing senescent cells with radiation, ultrasound, toxins, antibodies, and antibody-toxin conjugates, which systems include senescent cell-surface proteins for use in targeting of therapeutic molecules.

The selective killing of senescent cells has proven impractical in mammals other than genetically-modified laboratory research animals. Currently-available systems and methods exhibit substantial systemic toxicity, inadequate targeting of cells of interest, and a lack of adequate safety features. These shortcomings in the art have hampered the development of safe and effective therapies for the treatment of certain cancers and for slowing the effects of aging.

### SUMMARY OF THE DISCLOSURE

The present disclosure is based upon the discovery that a cell, such as a cell that is associated with aging, a disease, and/or another condition (collectively, “a target cell”), can be selectively killed, in a target cell-specific manner, without the need for targeted delivery of a therapeutic agent to the target cell. The expression constructs, systems, and methods described herein overcome safety and efficacy concerns that are associated with existing technologies that employ targeted delivery of therapeutic agents, which technologies have yielded limited therapeutic benefit to patients in need thereof.

As described herein, the present disclosure provides expression cassettes, systems, and methods for inducing, in a target cell-specific manner, the expression of a nucleic acid that encodes a protein that, when produced in a cell, reduces or eliminates the growth and/or survival of a cell, such as a cell that is associated with aging, disease, and/or other condition.

The expression cassettes, systems, and methods described herein exploit the unique transcription regulatory machinery that is intrinsic to certain cells that are associated with age (such as senescent cells), disease (such as cancers, infections diseases, and bacterial diseases), as well as other conditions, which transcription regulatory machinery is not operative, or exhibits substantially reduced activity, in a normal cell (i.e., “a non-target cell”) that is not associated with aging, disease, or other condition.

The presently-disclosed expression cassettes, systems, and methods achieve a high degree of target cell specificity as a consequence of intracellular functionality that is provided by, and unique to, the target cell, which intracellular functionality is not provided by, or is substantially reduced in, a normal, non-target cell. Thus, the presently disclosed systems and methods employ nucleic acid delivery vectors that are non-specific with respect to the cell type to which the nucleic acid is delivered and, indeed, the vectors described herein need not be configured for target cell-specific delivery of a nucleic acid (e.g., an expression cassette) to achieve target cell specificity and, consequently, the therapeutically effective reduction, prevention, and/or elimination in the growth and/or survival of a target cell.

Within certain embodiments, the present disclosure provides expression constructs for the targeted production of therapeutic proteins within a target cell, such as a cell that is associated with aging, disease, and/or another condition. The expression constructs disclosed herein comprise: (1) a transcriptional promoter that is activated in response to one or more factors each of which is produced within a target cell and (2) a nucleic acid that is operably linked to and under regulatory control of the transcriptional promoter, wherein the nucleic acid encodes a therapeutic protein that can reduce, prevent, and/or eliminate the growth and/or survival of a cell, including the target cell.

Within certain aspects of these embodiments, the transcriptional promoter is activated in a target cell that is associated with a disease, condition, or age but is not activated in a normal mammalian cell that is not associated with the disease, condition, or aging. Target cell-specific transcriptional activation is achieved by the action of one or more factors that are produced in the target cell but not produced in a normal mammalian cell, including a normal human cell, such as normal skeletal myoblasts, normal adipose cells, normal cells of the eye, normal brain cells, normal liver cells, normal colon cells, normal lung cells, normal pancreas cells, and/or normal heart cells, which normal cells are not associated with the disease, condition, or aging.

Within other aspects of these embodiements, the target cell can be a mammalian cell or a bacterial cell. Target mammalian cells can include human cells such as senescent cells, cancer cells, precancerous cells, dysplastic cells, and cells that are infected with an infectious agent.

In certain aspects of these embodiments wherein the human target cell is a senescent cell, the transcriptional promoter can include the p16INK4a/CDKN2A transcriptional promoter, which is responsive to activation by transcription factors such as SP1, ETS1, and/or ETS2. In other aspects of these embodiments wherein the human target cell is a senescent cell, the transcriptional promoter can include the p21/CDKN1A transcriptional promoter, which is responsive to p53/TP53. In a target cell, such as a senescent cell, transcriptional promoters induce the expression of a nucleic acid that encodes a therapeutic protein such as, for example, CASP3, CASP8, CASP9, BAX, DFF40, HSV-TK, and cytosine deaminase as well as inducible variants of CASP3, CASP8, CASP9, BAX, DFF40, HSV-TK, and cytosine deaminase which therapeutic protein reduces, prevents, and/or eliminates the growth and/or survival of the senescent cell, such as, for example, by inducing cell death in the senescent cell via a cellular process including apoptosis. Other therapeutic proteins may be employed that reduce, prevent, and/or eliminate the growth and/or survival of a senescent cell by, for example, inducing cell death via a cellular process including necrosis/necroptosis, autophagic cell death, endoplasmic reticulum-stress associated cytotoxicity, mitotic catastrophe, paraptosis, pyroptosis, pyronecrosis, and entosifs.

In other aspects of these embodiments wherein the human target cell is a cancer cell, such as a brain cancer cell, a prostate cancer cell, a lung cancer cell, a colorectal cancer cell, a breast cancer cell, a liver cancer cell, a hematologic cancer cell, and a bone cancer cell, the transcriptional promoter can include the p21^{cip1/waf1} promoter, the p27^{kip1} promoter, the p57^{kip}2 promoter, the TdT promoter, the Rag-1 promoter, the B29 promoter, the Blk promoter, the CD19 promoter, the BLNK promoter, and/or the λ5 promoter, which transcriptional promoter is responsive to activation by one or more transcription factors such as an EBF3, O/E-1, Pax-5, E2A, p53, VP16, MLL, HSF1, NF-IL6, NFAT1, AP-1, AP-2, HOX, E2F3, and/or NF-κB transcription factor, and which transcriptional activation induces the expression of a nucleic acid that encodes a therapeutic protein such as, for example, CASP3, CASP8, CASP9, BAX, DFF40, HSV-TK, or cytosine deaminase as well as inducible variants of CASP3, CASP8, CASP9, BAX, DFF40, HSV-TK, and cytosine deaminase which therapeutic protein reduces, prevents, and/or eliminates the growth and/or survival of the senescent cell, such as, for example, by inducing cell death in the senescent cell via a cellular process including apoptosis. Other therapeutic proteins may be employed that reduce, prevent, and/or eliminate the growth and/or survival of a senescent cell by, for example, inducing cell death via a cellular process including necrosis/necroptosis, autophagic cell death, endoplasmic reticulum-stress associated cytotoxicity, mitotic catastrophe, paraptosis, pyroptosis, pyronecrosis, and entosifs.

In still further aspects of these embodiments wherein the target cell is a human cell that is infected with an infectious agent, such as a virus, including, for example, a herpes virus, a polio virus, a hepatitis virus, a retrovirus virus, an influenza virus, and a rhino virus, or the target cell is a bacterial cell, the transcriptional promoter can be activated by a factor that is expressed by the infectious agent or bacterial cell, which transcriptional activation induces the expression of a nucleic acid that encodes a therapeutic protein such as, for example, CASP3, CASP8, CASP9, BAX, DFF40, HSV-TK, and cytosine deaminase as well as inducible variants of CASP3, CASP8, CASP9, BAX, DFF40, HSV-TK, and cytosine deaminase which therapeutic protein reduces, prevents, and/or eliminates the growth and/or survival of the senescent cell, such as, for example, by inducing cell death in the senescent cell via a cellular process including apoptosis. Other therapeutic proteins may be employed that reduce, prevent, and/or eliminate the growth and/or survival of a senescent cell by, for example, inducing cell death via a cellular process including necrosis/necroptosis, autophagic cell death, endoplasmic reticulum-stress associated cytotoxicity, mitotic catastrophe, paraptosis, pyroptosis, pyronecrosis, and entosifs.

Within other embodiments, the present disclosure provides systems for the targeted production of a therapeutic protein within a target cell. These systems comprise a vector that is capable of delivering a nucleic acid to a cell, including a target cell as well as a non-target cell, wherein the vector comprises an expression construct for the targeted production of a therapeutic protein within a target cell (e.g., a cell that is associated with age, disease, or other condition) but not within a non-target cell, wherein the expression construct comprises a transcriptional promoter that is activated in response to one or more factors each of which is produced within said target cell; and a nucleic acid that is operably linked to and under regulatory control of the transcriptional promoter, wherein the nucleic acid encodes a therapeutic protein that can reduce, prevent, and/or eliminate the growth and/or survival of a cell in which it is produced, including a target cell.

Within further aspect of these embodiments, the system further comprises one or more safety features that permit additional control over the expression of the nucleic acid within the expression construct or the functionality of a therapeutic protein encoded by the nucleic acid such as, for example, by requiring the contacting of a target cell with a chemical or biological compound that, in addition to the intracellular factor that promotes transcriptional activation of the promoter within the expression construct or promotes the functionality of the therapeutic protein, such as by promoting the dimerization of as well as inducible variants of CASP3, CASP8, CASP9, BAX, DFF40, HSV-TK, and cytosine deaminase.

A further safety element that may be employed in the expression constructs and systems of the present disclosure includes a tamoxifen-inducible Cre construct using Life Technologies Gateway Cloning Vector System employing a pDEST26 plasmid for mammalian expression. For example, a fusion protein of Cre and estrogen receptor can be constitutively expressed and induced upon the addition of tamoxifen, which permits activated Cre to re-orient the transcriptional promoter, thereby expressing the therapeutic protein.

Within yet other aspects of these embodiments, the system may further comprise a nucleic acid that encodes a detectable marker, such as a bioluminescent marker, thereby allowing the identification of cells that express the therapeutic protein and, in the case of an inducible therapeutic protein such as an inducible CASP3, CASP8, or CASP9, will be killed by the administration of a compound that promotes activity of the therapeutic protein, such as by inducing the dimerization of an inducible CASP3, CASP8, or CASP9.

Within further embodiments, the present disclosure provides methods for reducing, preventing, and/or eliminating the growth of a target cell, which methods comprise contacting a target cell with a system for the targeted production of a therapeutic protein within a target cell, wherein the system comprises a vector that is capable of delivering a nucleic acid to a cell, wherein the vector comprises an expression construct for the targeted production of a therapeutic protein within a target cell (e.g., a cell that is associated with age, disease, or other condition) but not within a non-target cell, wherein the expression construct comprises: (a) a transcriptional promoter that is activated in response to one or more factors each of which factors is produced within a target cell and (b) a nucleic acid that is operably linked to and under regulatory control of the transcriptional promoter, wherein the nucleic acid encodes a therapeutic protein that is produced upon expression of the nucleic acid and wherein production of the therapeutic protein in the target cell (i.e., the cell that is associated with age, disease, or other condition) reduces, prevents, and/or eliminates growth and/or survival of the target cell.

Within still further embodiments, the present disclosure provides methods for the treatment of an aging human or a human that is afflicted with a disease or another condition, wherein the aging, disease, or other condition is associated with a target cell within the human, the methods comprising administering to the human a system for the targeted production of a therapeutic protein within a target cell, wherein the system comprises a vector that is capable of delivering a nucleic acid to a cell, wherein the vector comprises an expression construct for the targeted production of a therapeutic protein within a target cell (e.g., a cell that is associated with age, disease, or other condition) but not within a non-target cell, wherein the expression construct comprises: (a) a transcriptional promoter that is activated in response to one or more factors each of which factors is produced within a target cell and (b) a nucleic acid that is operably linked to and under regulatory control of the transcriptional promoter, wherein the nucleic acid encodes a therapeutic protein that is produced upon expression of the nucleic acid and wherein production of the therapeutic protein in the target cell (i.e., the cell that is associated with age, disease, or other condition) reduces, prevents, and/or eliminates growth and/or survival of the target cell thereby slowing aging in the human and/or slowing, reversing, and/or eliminating the disease or condition in the human.

These and other related aspects of the present disclosure will be better understood in light of the following drawings and detailed description, which exemplify certain aspects of the various embodiments.

### BRIEF DESCRIPTION OF THE DRAWINGS

FIG. 1 is a map of a plasmid comprising an exemplary expression cassette wherein the promoter sequence is a p16 transcriptional promoter (from Baker et al., Nature 479(7372):232-67 (2011)) and the nucleic acid encoding a therapeutic protein encodes the inducible caspase 9 variant FC1s.dCasp9.

FIG. 2 is a map of an exemplary expression cassette wherein the promoter sequence is a p16 transcriptional promoter (from Baker et al., Nature 479(7372):232-67 (2011)) and the nucleic acid encoding a therapeutic protein encodes caspase 9 (non-inducible). This expression cassette comprises a further safety element, which includes a tamoxifen-inducible Cre construct.

FIG. 3 is a map of a plasmid comprising an exemplary expression cassette wherein the promoter sequence is a p16 transcriptional promoter (from Baker et al., Nature 479(7372):232-67 (2011)) and the nucleic acid encoding a therapeutic protein encodes caspase 9 (non-inducible). This embodiment comprises a further safety element, which includes a tamoxifen-inducible Cre construct using Life Technologies Gateway Cloning Vector System employing a pDEST26 plasmid for mammalian expression. A fusion protein of Cre and estrogen receptor is constitutively expressed and induced upon the addition of tamoxifenm which permits activated Cre to re-orient the p16-promoter, thereby expressing caspase 9.

FIG. 4 is a map of a plasmid comprising an exemplary expression cassette wherein the promoter sequence is a p16 transcriptional promoter (from Baker et al., Nature 479(7372):232-67 (2011)) and the nucleic acid encoding a therapeutic protein encodes the inducible caspase 9 variant FC1s.dCasp9. This embodiment comprises a further safety element, which includes a tamoxifen-inducible Cre construct using Life Technologies Gateway Cloning Vector System employing a pDEST26 plasmid for mammalian expression. A fusion protein of Cre and estrogen receetor is constitutively expressed and induced upon the addition of tamoxifenm which permits activated Cre to re-orient the p16-promoter, thereby expressing the inducible caspase 9 variant FC1s.dCasp9.

FIG. 5 is the nucleotide sequence of a p16 promoter as shown in FIGS. 1-4 and as described in Baker et al., Nature 479(7372):232-67 (2011) and in Wang et al., J Biol Chem 276(52):48655-61 (2001).

FIG. 6 is the high affinity p53 binding sequence from the human ConA Gene as described in Noureddine et al., PLoS Genet 5(5):e1000462 (2009)

FIG. 7 is the nucleotide sequence encoding human caspase9α (GeneID: 842, Validated mRNA Sequence NM_{—}001229).

FIG. 8 is the nucleotide sequence encoding human caspase3 pre-protein (GeneID: 836, Validate mRNA NM_{—}004346).

FIG. 9 is the nucleotide sequence encoding human DFFB protein (GeneID: 1677, Validated mRNA NM_001282669).

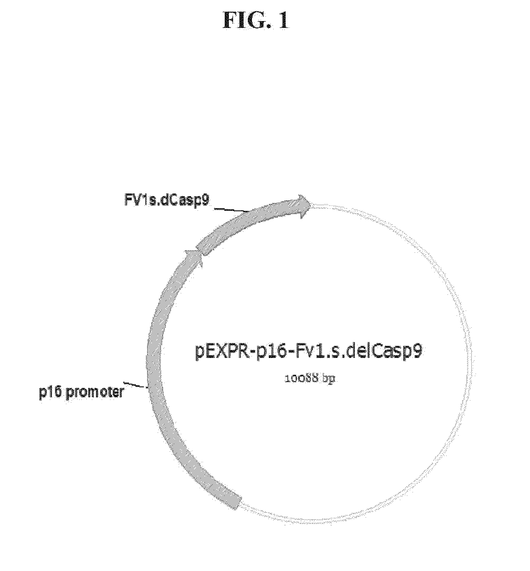

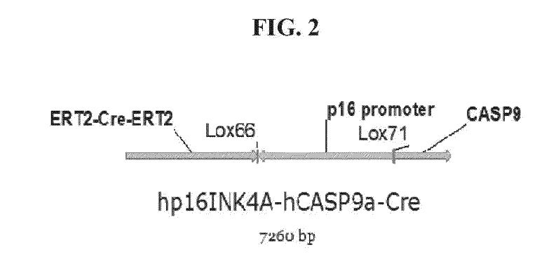

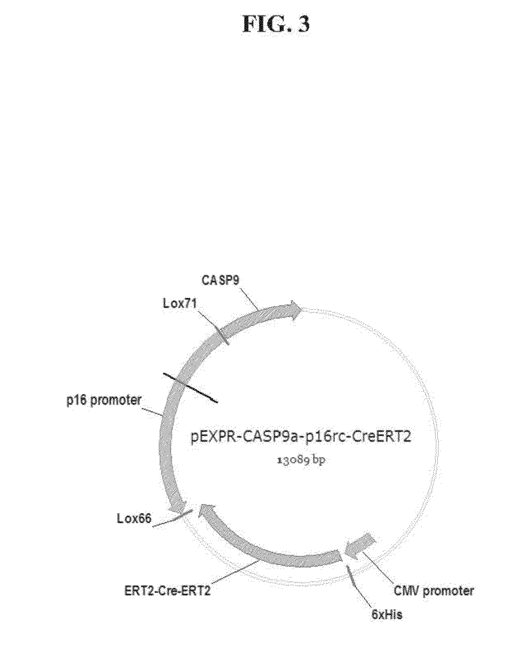

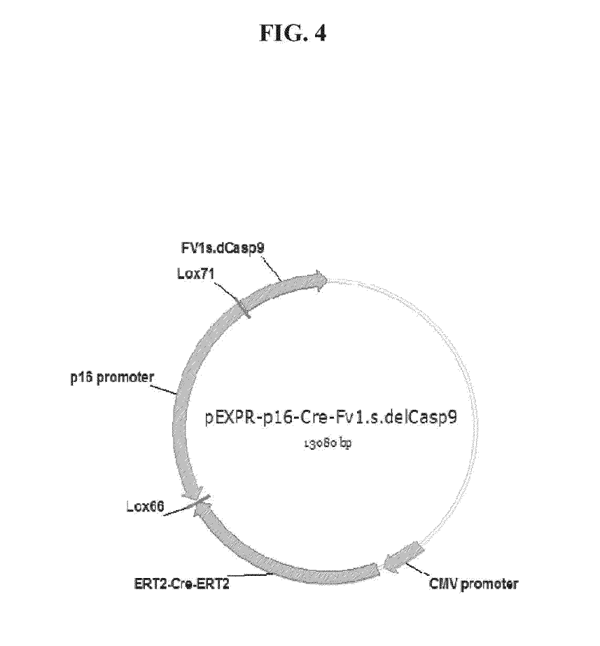

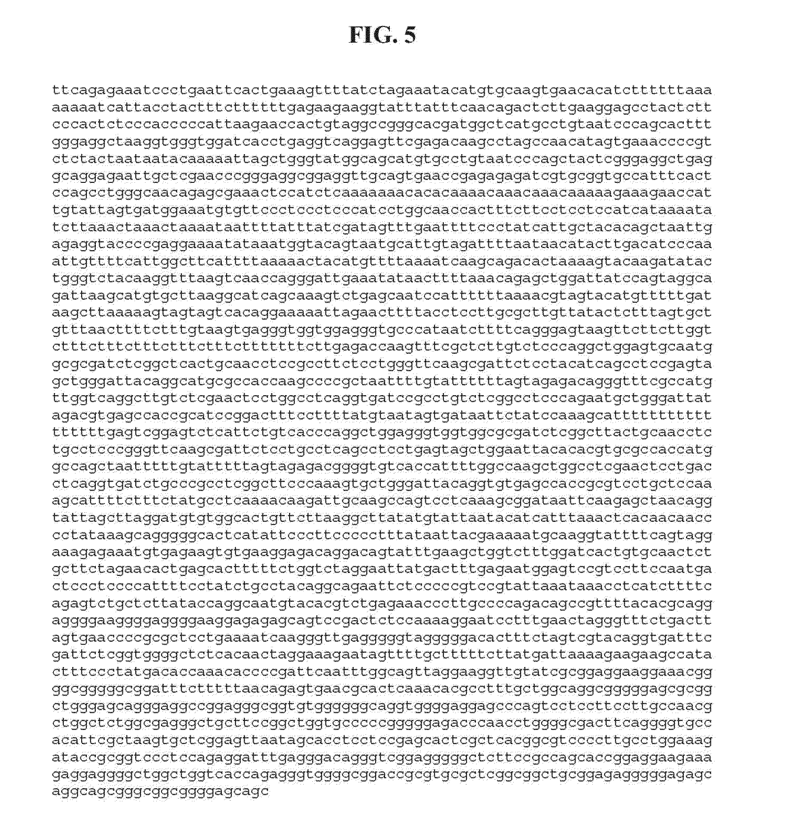

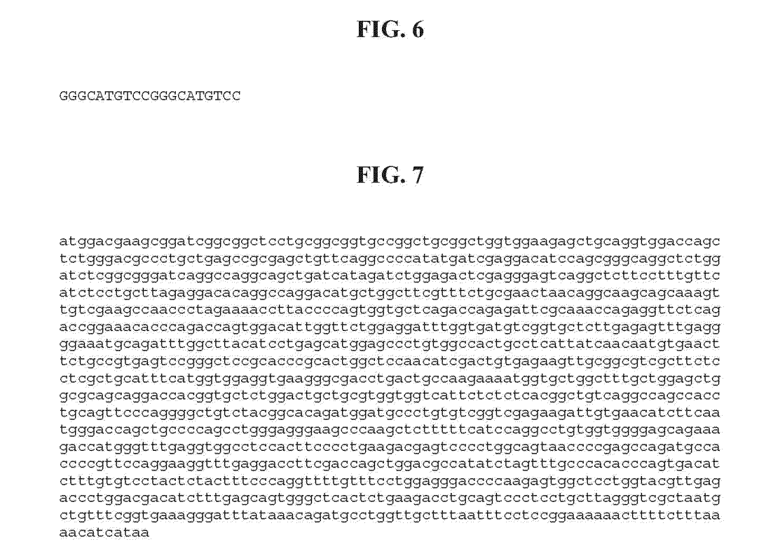

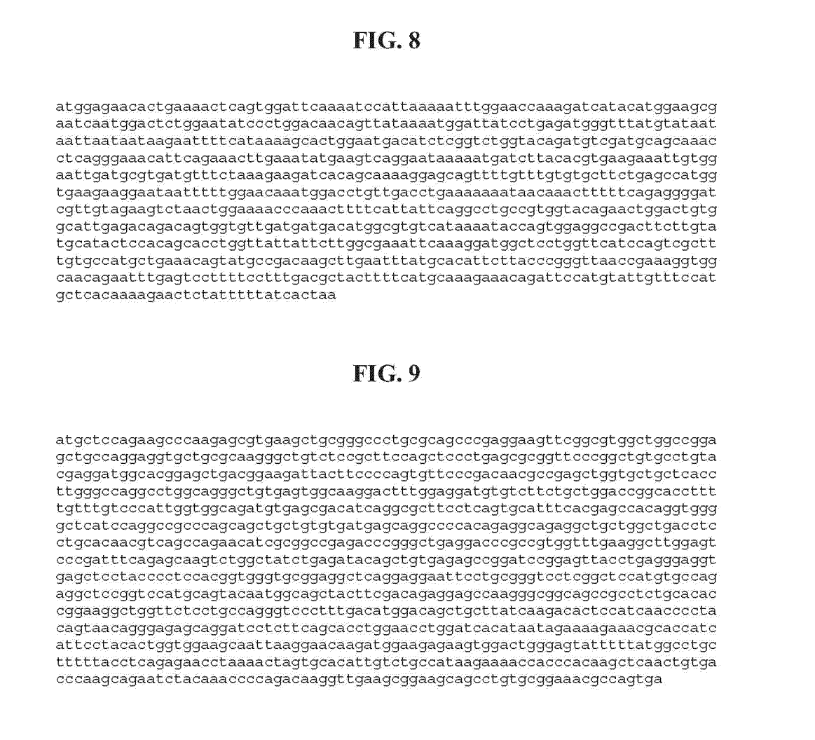

### DETAILED DESCRIPTION

The present disclosure provides expression cassettes, systems, and methods for the selective reduction, prevention, and/or elimination in the growth and/or survival of a cell that is associated with aging, disease, or another condition (collectively “a target cell”), which expression cassettes, systems, and methods overcome the safety and efficacy concerns that are associated with existing technologies that rely on targeted delivery of a therapeutic compound and, as a result of, for example, inefficient target cell delivery and/or off-target effects, have limited therapeutic benefit.

More specifically, the expression cassettes, systems, and methods disclosed herein exploit the cell-specific transcription regulatory machinery that is intrinsic to a target cell and, thereby, achieve a target cell-specific therapeutic benefit without the need for targeted-delivery of a therapeutic compound. These expression cassettes, systems, and methods permit the target cell-specific induction of expression of a nucleic acid that encodes a therapeutic protein, which protein can reduce, prevent, and/or eliminate the growth and/or survival of a cell in which it is produced.

Thus, the various embodiments that are provided by the present disclosure include:
(1) expression constructs for the targeted production of therapeutic proteins within a target cell, such as a cell that is associated with aging, disease, and/or another condition, the expression construct comprising:
   (a) a transcriptional promoter that is activated in response to one or more factors each of which is produced within a target cell and
   (b) a nucleic acid that is operably linked to and under regulatory control of the transcriptional promoter, wherein the nucleic acid encodes a therapeutic protein that can reduce, prevent, and/or eliminate the growth and/or survival of a cell, including the target cell;
(2) systems for the targeted production of a therapeutic protein within a target cell, the systems comprising:
   a vector that is capable of delivering a nucleic acid to a cell, including a target cell as well as a non-target cell,
      wherein the vector comprises an expression construct for the targeted production of a therapeutic protein within a target cell (e.g., a cell that is associated with age, disease, or other condition) but not within a non-target cell,
   the expression construct comprising:
      (a) a transcriptional promoter that is activated in response to one or more factors each of which is produced within said target cell; and
      (b) a nucleic acid that is operably linked to and under regulatory control of the transcriptional promoter,
         wherein the nucleic acid encodes a therapeutic protein that can reduce, prevent, and/or eliminate the growth and/or survival of a cell in which it is produced, including a target cell.
(3) methods for reducing, preventing, and/or eliminating the growth of a target cell, the methods comprising: contacting a target cell with a system for the targeted production of a therapeutic protein within a target cell,
   wherein the system comprises a vector that is capable of delivering a nucleic acid to a cell,
   wherein the vector comprises an expression construct for the targeted production of a therapeutic protein within a target cell (e.g., a cell that is associated with age, disease, or other condition) but not within a non-target cell,
   wherein the expression construct comprises:
      (a) a transcriptional promoter that is activated in response to one or more factors each of which factors is produced within a target cell and
      (b) a nucleic acid that is operably linked to and under regulatory control of the transcriptional promoter,
   wherein the nucleic acid encodes a therapeutic protein that is produced upon expression of the nucleic acid and
   wherein production of the therapeutic protein in the target cell (i.e., the cell that is associated with age, disease, or other condition) reduces, prevents, and/or eliminates growth and/or survival of the target cell.
(4) methods for the treatment of aging, disease, or other condition in a human, wherein aging, disease, or other condition is associated with a target cell, the methods comprising:
administering to the human a system for the targeted production of a therapeutic protein within a target cell,
   wherein the system comprises a vector that is capable of delivering a nucleic acid to a cell,
   wherein the vector comprises an expression construct for the targeted production of a therapeutic protein within a target cell (e.g., a cell that is associated with age, disease, or other condition) but not within a non-target cell,
   wherein the expression construct comprises:
      (a) a transcriptional promoter that is activated in response to one or more factors each of which factors is produced within a target cell and
      (b) a nucleic acid that is operably linked to and under regulatory control of the transcriptional promoter,
   wherein the nucleic acid encodes a therapeutic protein that is produced upon expression of the nucleic acid and
   wherein production of the therapeutic protein in the target cell (i.e., the cell that is associated with age, disease, or other condition) reduces, prevents, and/or eliminates growth and/or survival of the target cell thereby slowing aging in the human and/or slowing, reversing, and/or eliminating the disease or condition in the human.

These and other aspects of the present disclosure can be better understood by reference to the following non-limiting definitions.

### Definitions

As used herein, the term “transcriptional promoter” refers to a promoter is a region of DNA that initiates transcription of a particular gene. Promoters are located near the transcription start sites of genes, on the same strand and upstream on the DNA (towards the 3′ region of the anti-sense strand, also called template strand and non-coding strand). Promoters can be about 100-1000 base pairs long. For the transcription to take place, the enzyme that synthesizes RNA, known as RNA polymerase, must attach to the DNA near a gene. Promoters contain specific DNA sequences and response elements that provide a secure initial binding site for RNA polymerase and for proteins called transcription factors that recruit RNA polymerase. These transcription factors have specific activator or repressor sequences of corresponding nucleotides that attach to specific promoters and regulate gene expressions. The process is more complicated, and at least seven different factors are necessary for the binding of an RNA polymerase II to the promoter. Promoters represent critical elements that can work in concert with other regulatory regions (enhancers, silencers, boundary elements/insulators) to direct the level of transcription of a given gene.

Eucaryotic transcriptional promoters comprise a number of essential elements, which collectively constitute a core promoter (i.e., the minimal portion of a promoter that is required to initiate transcription). Those elements include (1) a transcription start site (TSS), (2) an RNA polymerase binding site (in particular an RNA polymerase II binding site in a promoter for a gene encoding a messenger RNA), (3) a general transcription factor binding site (e.g., a TATA box having a consensus sequence TATAAA, which is a binding site for a TATA-binding protein (TBP)), (4) a B recognition element (BRE), (5) a proximal promoter of approximately 250 by that contains regulatory elements, (6) transcription factor binding sites (e.g., an E-box having the sequence CACGTF, which is a binding site for basic helix-loop-helix (bHLH) transcription factors including BMAL11-Clock nad cMyc), and (7) a distal promoter containing additional regulatory elements. As used herein, the term “transcriptional promoter” is distinct from the term “enhancer,” which refers to a regulatory element that is distant from the transcriptional start site.

Eucaryotic promoters are often categorized according to the following classes: (1) AT-based class, (2) CG-based class, (3) ATCG-compact class, (4) ATCG-balanced class, (5) ATCG-middle class, (6) ATCG-less class, (7) AT-less class, (8) CG-spike class, (9) CG-less class, and (10) ATspike class. See, Gagniuc and Ionescu-Tirgoviste, BMC Genomics 13:512 (2012).

Eucaryotic promoters can be “unidirectional” or “bidirectional.” Unidirectional promoters regulate the transcription of a single gene and are characterized by the presence of a TATA box. Bidirectional promoters are short (<1 kbp), intergenic regions of DNA between the 5′ ends of genes in a bidirectional gene pair (i.e., two adjacent genes coded on opposite strands having 5′ ends oriented toward one another. Bidirectional genes are often functionally related and because they share a single promoter, can be co-regulated and co-expressed. Unlike unidirectional promoters, bidirectional promoters do not contain a TATA box but do contain GpC islands and exhibit symmetry around a midpoint of dominant Cs and As on one side and Gs and Ts on the other. CCAAT boxes are common in bidirectional promoters as are NRF-1, GABPA, YY1, and ACTACAnnTCCC motifs.

Transcriptional promoters often contain two or more transcription factor binding sites. Thus, the efficient expression of a nucleic acid that is downstream of a promoter having multiple transcription factor binding sites typically requires the cooperative action of multiple transcription factors. Accordingly, the specificity of transcriptional regulation, and hence expression of an associated nucleic acid, can be increased by employing transcriptional promoters having two or more transcription factor binding sites.

As used herein, the term “transcription factor” refers to sequence-specific DNA-binding factors that bind to specific sequences within a transcriptional promoter thereby regulating the transcription of a nucleic acid that is in operable proximity to and downstream of the promoter. Transcription factors include activators, which promote transcription, and repressors, which block transcription by preventing the recruitment or binding of an RNA polymerase. Transcription factors typically contain (1) one or more DNA-binding domains (DBDs), which facilitate sequence specific binding to a cognate transcription factor binding site (a/k/a response element) within a transcriptional promoter; (2) one or more signal-sensing domains (SSDs), which includes ligand binding domains that are responsive to external signals; and (3) one or more transactivation domains (TADs), which contain binding sites for other proteins, including transcription coregulators.

As used herein, the term “transcription factor” refers exclusively to those factors having one or more DBDs and is not intended to include other regulatory proteins such as coactivators, chromatin remodelers, histone acetylases, deacetylases, kinases, and methylases, which no not contain DBDs.

Of the approximately 2,600 human proteins that contain DNA-binding domains, the majority are believed to be transcription factors. Transcription factors are categorized according to structural features of the DNA-binding domain, which include basic helix-loop-helix domains, basic-leucine zipper (bZIP domains), C-terminal effector domains of bipartite response regulators, GCC box domains, helix-turn-helix domains, homeodomains, lambda repressor-like domains, serum response factor-like (srf-like) domains, paired box domains, winged helix domains, zinc finger domains, multi-Cys₂His₂ zinc finger domains, Zn₂Cys₆ domains, and Zn₂Cys₈ nuclear receptor zinc finger domains.

Many transcription factors are either tumor suppressors or oncogenes, and, thus, mutations within and the aberrant expression of such transcription factors is associated with some cancers and other diseases and conditions. For example, transcription factors within (1) the NF-kappaB family, (2) the AP-1 family, (3) the STAT family, and (4) the steroid receptor family have been implicated in the neurodevelopmental disorder Rett syndrome (the MECP2 transcription factor), diabetes (hepatocyte nuclear factors (HNFs) and insulin promoter factor-1 (IPF1/Pdx1)), developmental verbal dyspraxia (the FOXP2 transcription factor), autoimmune diseases (the FOXP3 transcription factor), Li-Raumeni syndrome (the p53 tumor suppressor), and multiple cancers (the STAT and HOX family of transcription factors). Clevenger, Am. J. Pathol. 165(5):1449-60 (2004); Carrithers et al., Am J Pathol 166(1):185-196 (2005); Herreros-Villanueve et al., World J Gastroenterology 20(9):2247-2254 (2014); and Campbell et al., Am J Pathol 158(1):25-32 (2001).

Olsson et al., Oncogene 26(7):1028-37 describe the upregulation of the transcription factor E2F3, which is a key regulator of the cell cycle, in human bladder and prostate cancers. Cantile et al., Curr Med Chem 18(32):4872-84 describe the upregulation of HOX genes in urogenital cancers; Cillo et al., Int J. Cancer 129(11):2577-87 (2011) describe the upregulation of HOX genes in hepatocellular carcinoma; Cantile et al., Int J. Cancer 125(7):1532-41 (2009) describe HOX D13 expression across 79 tumor tissue types; Cantile et al., J Cell Physiol 205(2):202-10 (2005) describe upregulation of HOX D expression in prostate cancers; Cantile et al., Oncogene 22(41):6462-8 (2003) describe the hyperexpression of locus C genes in the HOX network in human bladder transitional cell carcinomas; Morgan et al., BioMed Central 14:15 (2014), describe HOX transcription factors as targets for prostate cancer; and Alharbi et al., Leukemia 27(5):1000-8 (2013) describe the role of HOXC genes in hematopoiesis and acute leukemia.

The AP-2 family includes five transcription factors that can act as both repressors and activators. AP-2γ regulates cancer cell survival by blocking p53 activation of the p21 CIP gene. High levels of AP-2γ are associated with poor prognosis in breast cancer. Gee et al., J Pathol 217(1):32-41 (2009) and Williams et al., EMBO J 28(22):3591-601 (2009). A further transcription factor that promotes cell survival are the forkhead transcription factors (FOX), which can promote the expression of proteins involved in drug resistance and also block programmed cell death and may therefore protect cancer cells from chemotherapeutic drugs. Gomes et al., Chin J. Cancer 32(7):365-70 (2013) describe the role of FOXO3a and FOXM1 in carcinogenesis and drug resistance.

Transcription factors can bind to promoters as well as to enhancers. As used in the present disclosure, the term transcription factor refers to the subset of transcription factors that bind to transcription factor binding sites within a promoter and excludes those factors that bind to enhancer sequences. Transcription factors can also upregulate or downregulate the expression of an associated nucleic acid. The present disclosure employs transcriptional promoters having transcription factor binding sites for transcription factors that promote rather than inhibit expression and therefore cause the upregulation in the expression of an associated nucleic acid. Such transcription factors that upregulate nucleic acid expression include, for example and not limitation, transcription factors that (1) stabilize RNA polymerase binding to its cognate binding site, (2) recruit coactivator or corepressor proteins to a transcription factor DNA complex, and/or (3) catalyze the acetylation of histone proteins (or recruit one or more other proteins that catalyze the acetylation of histone proteins). Such histone acetyltransferase (HAT) activity reduces the affinity of histone binding to DNA thereby making the DNA more accessible for transcription.

As used herein, the term “necrosis” refers to a process leading to cell death that occurs when a cell is damaged by an external force, such as poison, a bodily injury, an infection, or loss of blood supply. Cell death from necrosis causes inflammation that can result in further distress or injury within the body. As used herein, the term “apoptosis” refers to a process leading to cell death in which a programmed sequence of events leads to the elimination of cells without releasing harmful substances. Apoptosis plays a crucial role in developing and maintaining the health of the body by eliminating old cells, unnecessary cells, and unhealthy cells. Apoptosis is mediated by proteins produced by suicide genes, including the caspase proteins, which break down cellular components needed for survival and induce the production of DNAses, which destroy nuclear DNA.

As used herein, the term “suicide gene” refers to a class of genes that produce proteins that induce p53-mediated apoptotic cell killing. Suicide genes that can be employed in the expression constructs and systems of the present disclosure include the caspases, CASP3, CASP8, CASP9, BAX, DFF40, Herpes Simplex Virus Thymidine Kinase (HSV-TK), and cytosine deaminase and inducible variants of CASP3, CASP8, CASP9, BAX, DFF40, Herpes HSV-TK, and cytosine deaminase.

The presently disclosed expression constructs and systems are used in methods for the treatment of aging, cancer infectious disease, bacterial infections, and/or other conditions as well as in methods for the killing of cells that are associated with aging, cancer, infectious disease, bacterial infections, and/or other conditions and employ a therapeutic protein that reduces the growth and/or proliferation of a target cell. In certain embodiments, the therapeutic protein can be expressed by a suicide gene, which encodes CASP3, CASP8, CASP9, BAX, DFF40, HSV-TK, or cytosine deaminase as well as an inducible variants of CASP3, CASP8, CASP9, BAX, DFF40, HSV-TK, or cytosine deaminase. The expression cassettes and systems can also be used in conjunction with conventional chemotherapeutics to enhance the effectiveness of therapeutic regimen for the treatment of aging, cancers, infectious diseases, bacterial infections, and other diseases and conditions.

The practice of the present disclosure will employ, unless indicated specifically to the contrary, conventional methodology and techniques that are in common use in the fields of virology, oncology, immunology, microbiology, molecular biology, and recombinant DNA, which methodology and techniques are well known by and readily available to those having skill of the art. Such methodology and techniques are explained fully in laboratory manuals as well as the scientific and patent literature. See, e.g., Sambrook, et al., “Molecular Cloning: A Laboratory Manual” (2nd Edition, Cold Spring Harbor Laboratory Press, Cold Spring Harbor, N.Y., 1989); Maniatis et al., “Molecular Cloning: A Laboratory Manual” (1982); “DNA Cloning: A Practical Approach, vol. I & II” (Glover, ed.); “Oligonucleotide Synthesis” (Gait, ed., 1984); Ausubel et al. (eds.), “Current Protocols in =Molecular Biology” (John Wiley & Sons, 1994); “Nucleic Acid Hybridization” (Hames & Higgins, eds., 1985); “Transcription and Translation” (Hames & Higgins, eds., 1984); “Animal Cell Culture” (Freshney, ed., 1986); and Perbal, “A Practical Guide to Molecular Cloning” (1984). All publications, patents and patent applications cited herein, whether supra or infra, are hereby incorporated by reference in their entirety.

### Systems and Expression Constructs for Reducing, Preventing, and/or Eliminating the Growth and/or Survival of a Target Cell

Within certain embodiments, the present disclosure provides expression constructs and systems comprising a delivery vector and an expression construct for achieving a target cell specific reduction, prevention, and/or elimination in the growth and/or survival of the target cell.

Systems

Systems of the present disclosure comprise (1) a vector that is capable of non-specific delivery of a nucleic acid to a cell, whether that cell is a target cell or a non-target cell, and (b) an expression construct comprising a target cell specific transcriptional promoter and a nucleic acid that encodes a therapeutic protein, which expression constructs achieve the target cell specific production of a therapeutic protein. The systems disclosed herein will find utility in a broad range of therapeutic applications in which it is desireable to effectuate the growth or survival characteristics of a target cell, such as a cell that is associated with aging, disease, or another condition, but, at the same time, to not effectuate the growth or survival characteristics of a normal, a non-target cell that is not associated with aging, disease, or another condition.

The present disclosure provides systems for effectuating the growth and/or survival of a broad range of cells that are associated with aging, disease, or other conditions that similarly comprises (1) a non-specific nucleic acid delivery vector and (2) an expression construct comprising (a) a target cell specific transcriptional promoter and (b) a nucleic acid that encodes a therapeutic protein. Each of these aspects of the presently disclosed systems are described in further detail herein.

Within certain embodiments, provided herein are systems for effectuating the growth and/or survival of target cells, which systems comprise: (1) a non-specific nucleic acid delivery vector and (2) an expression construct comprising: (a) a transcriptional promoter, which transcriptional promoter is activated in target cells but not in normal, non-target cells, and (b) a nucleic acid that is under the control of the transcriptional promoter, which nucleic acid encodes a therapeutic protein that can reduce, prevent, and/or eliminate the growth and/or survival of a target cell, for example by inducing a mechanism of programmed cell death in a cell in which it is produced. Thus, these systems achieve the selective killing of target cells by exploiting transcriptional machinery that is produced in, and intrinsic to, target cells; without the use of toxins and in the absence of target cell specific delivery of the expression construct.

In certain aspects of these embodiments wherein the human target cell is a senescent cell, the transcriptional promoter can include at least a transcription factor binding site (i.e., a response element) of p16INK4a/CDKN2A as described in Wang et al., J. Biol. Chem. 276(52):48655-61 (2001), which transcriptional promoter is responsive to activation by a factor such as SP1, ETS1, and ETS2. The transcriptional promoter can also include at least a transcription factor binding site (i.e., a response element) of p21/CDKN1A, which transcriptional promoter is responsive to activation by a factor such as p53/TP53. Transcriptional activation induces the expression of a nucleic acid that encodes a therapeutic protein such as CASP3, CASP8, CASP9, DFF40, BAX, HSV-TK, or carbonic anhydrase or an inducible variant of CASP3, CASP8, CASP9, BAX, DFF40, HSV-TK, or cytosine deaminase.

In other aspects of these embodiments wherein the human target cell is a cancer cell, such as a brain cancer cell, a prostate cancer cell, a lung cancer cell, a colorectal cancer cell, a breast cancer cell, a liver cancer cell, a hematologic cancer cell, and a bone cancer cell, the transcriptional promoter can include at least a transcription factor binding site (i.e., a response element) of the p21^{cip1/waf1} promoter, the p27^{kip1} promoter, the p57^{kip2} promoter, the TdT promoter, the Rag-1 promoter, the B29 promoter, the Blk promoter, the CD19 promoter, the BLNK promoter, and/or the λ5 promoter, which transcriptional promoter is responsive to activation by one or more transcription factors such as an EBF3, O/E-1, Pax-5, E2A, p53, VP16, MLL, HSF1, NF-IL6, NFAT1, AP-1, AP-2, HOX, E2F3, and/or NF-κB transcription factor, and which transcriptional activation induces the expression of a nucleic acid that encodes a therapeutic protein such as, for example, CASP3, CASP8, CASP9, BAX, DFF40, HSV-TK, or cytosine deaminase or an inducible variant of CASP3, CASP8, CASP9, BAX, DFF40, HSV-TK, or cytosine deaminase which therapeutic protein reduces, prevents, and/or eliminates the growth and/or survival of the cancer cell, such as, for example, by inducing cell death in the senescent cell via a cellular process including apoptosis. Other therapeutic proteins may be employed that reduce, prevent, and/or eliminate the growth and/or survival of a cancer cell by, for example, inducing cell death via a cellular process including necrosis/necroptosis, autophagic cell death, endoplasmic reticulum-stress associated cytotoxicity, mitotic catastrophe, paraptosis, pyroptosis, pyronecrosis, and entosifs.

In still further aspects of these embodiments wherein the target cell is a human cell that is infected with an infectious agent, such as a virus, including, for example, a herpes virus, a polio virus, a hepatitis virus, a retrovirus, an influenza virus, and a rhino virus, or the target cell is a bacterial cell, the transcriptional promoter can be activated by a factor that is expressed by the infectious agent or bacterial cell, which transcriptional activation induces the expression of a nucleic acid that encodes a therapeutic protein such as, for example, CASP3, CASP8, CASP9, BAX, DFF40, HSV-TK, or cytosine deaminase or an inducible variant of CASP3, CASP8, CASP9, BAX, DFF40, HSV-TK, or cytosine deaminase which therapeutic protein reduces, prevents, and/or eliminates the growth and/or survival of the senescent cell, such as, for example, by inducing cell death in the senescent cell via a cellular process including apoptosis. Other therapeutic proteins may be employed that reduce, prevent, and/or eliminate the growth and/or survival of a senescent cell by, for example, inducing cell death via a cellular process including necrosis/necroptosis, autophagic cell death, endoplasmic reticulum-stress associated cytotoxicity, mitotic catastrophe, paraptosis, pyroptosis, pyronecrosis, and entosifs.

Each of these aspects of the presently disclosed systems are described in further detail herein.

Non-specific Nucleic Acid Delivery Vectors

The systems of the present disclosure achieve target cell specificity by exploiting transcriptional machinery that is unique to a target cell. Thus, the systems described herein employ nucleic acid delivery vectors that can be readily adapted for the non-specific delivery of expression constructs to a cell, including but not limited to a target cell.

A wide variety of both non-viral and viral nucleic acid delivery vectors are well known and readily available in the art and may be adapted for use for the non-specific cellular delivery of the expression constructs disclosed herein. See, for example, Elsabahy et al., Current Drug Delivery 8(3):235-244 (2011) for a general description of viral and non-viral nucleic acid delivery methodologies. The successful delivery of a nucleic acid into mammalian cells relies on the use of efficient delivery vectors. Viral vectors exhibit desireable levels of delivery efficiency, but often also exhibit undesireable immunogenicity, inflammatory reactions, and problems associated with scale-up, all of which can limit their clinical use. The ideal vectors for the delivery of a nucleic acid are safe, yet ensure nucleic acid stability and the efficient transfer of the nucleic acid to the appropriate cellular compartments.

Non-limiting examples of non-viral and viral nuclic acid delivery vectors are described herein and disclosed in scientific and patent literature. More specifically, the presently disclosed systems may employ one or more liposomal vectors, viral vectors, nanoparticles, polyplexesm dendrimers, each of which has been developed for the non-specific delivery of nucleic acids, can be adapted for the non-specific delivery of the expression constructs described herein, and can be modified to incorporate one or more agents for promoting the targeted delivery of a system to a target cell of interest thereby enhancing the target cell specificity of the presently disclosed systems.

1. Liposomal Vectors

An expression cassette may be incorporated within and/or associated with a lipid membrane, a lipid bi-layer, and/or a lipid complex such as, for example, a liposome, a vesicle, a micelle and/or a microsphere. Suitable methodology for preparing lipid-based delivery systems that may be employed with the expression constructs of the present disclosure are described in Metselaar et al., Mini Rev. Med. Chem. 2(4):319-29 (2002); O'Hagen et al., Expert Rev. Vaccines 2(2):269-83 (2003); O'Hagan, Curr. Durg Targets Infect. Disord. 1(3):273-86 (2001); Zho et al., Biosci Rep. 22(2):355-69 (2002); Chikh et al., Biosci Rep. 22(2):339-53 (2002); Bungener et al., Biosci. Rep. 22(2):323-38 (2002); Park, Biosci Rep. 22(2):267-81 (2002); Ulrich, Biosci. Rep. 22(2):129-50; Lofthouse, Adv. Drug Deliv. Rev. 54(6):863-70 (2002); Zhou et al., J. Immunother. 25(4):289-303 (2002); Singh et al., Pharm Res. 19(6):715-28 (2002); Wong et al., Curr. Med. Chem. 8(9):1123-36 (2001); and Zhou et al., Immunomethods 4(3):229-35 (1994). Midoux et al., British J. Pharmacol 157:166-178 (2009) describe chemical vectors for the delivery of nucleic acids including polymers, peptides and lipids. Sioud and Sorensen, Biochem Biophys Res Commun 312(4):1220-5 (2003) describe cationic liposomes for the delivery of nucleic acids.

Due to their positive charge, cationic lipids have been employed for condensing negatively charged DNA molecules and to facilitate the encapsulation of DNA into liposomes. Cationic lipids also provide a high degree of stability to liposomes. Cationic liposomes interact with a cell membrane and are taken up by a cell through the process of endocytosis. Endosomes formed as the results of endocytosis, are broken down in the cytoplasm thereby releasing the cargo nucleic acid. Because of the inherent stability of cationic liposomes, however, transfection efficiencies can be low as a result of lysosomal degradation of the cargo nucleic acid.

Helper lipids (such as the electroneutral lipid DOPE and L-a-dioleoyl phosphatidyl choline (DOPC)) can be employed in combination with cationic lipids to form liposomes having decreased stability and, therefore, that exhibit improved transfection efficiencies. These electroneutral lipids are referred to as fusogenic lipids. See, Gruner et al., Biochemistry 27(8):2853-66 (1988) and Farhood et al., Biochim Biophys Acta 1235(2):289-95 (1995). DOPE forms an HII phase structure that induces supramolecular arrangements leading to the fusion of a lipid bilayer at a temperature greater than 5° C. to 10° C. The incorporation of DOPE into liposomes also helps the formation of HII phases that destabilize endosomal membranes.

Cholesterol can be employed in combination with DOPE liposomes for applications in which a liposomal vector is administered intravenously. Sakurai et al., Eur J Pharm Biopharm 52(2):165-72 (2001). The presence of one unsaturation in the acyl chain of DOPE is a crucial factor for membrane fusion activity. Talbot et al., Biochemistry 36(19):5827-36 (1997).

Fluorinated helper lipids having saturated chains, such as DF4C11PE (rac-2,3-Di[11-(F-butyl)undecanoyl) glycero-1-phosphoethanolamine) also enhance the transfection efficiency of lipopolyamine liposomes. Boussif et al., J Gene Med 3(2):109-14 (2001); Gaucheron et al., Bioconj Chem 12(6):949-63 (2001); and Gaucheron et al., J Gene Med 3(4):338-44 (2001).

The helper lipid 1,2-dioleoyl-3-trimethylammonium-propane (DOTAP) enhances efficient of in vitro cell transfection as compared to DOPE lipoplexes. Prata et al., Chem Commun 13:1566-8 (2008). Replacement of the double bond of the oleic chains of DOPE with a triple bond as in Distear-4-ynoyl L-a-phosphatidylethanolamine [DS(9-yne)PE] has also been shown to produce more stable lipoplexes. Fletcher et al., Org Biomol Chem 4(2):196-9 (2006).

Amphiphilic anionic peptides that are derived from the N-terminal segment of the HA-2 subunit of influenza virus haemagglutinin, such as the IFN7 (GLFEAIEGFIENGWEGMIDGWYG) and E5CA (GLFEAIAEFIEGGWEGLIEGCA) peptides, can be used to increase the transfection efficiency of liposomes by several orders of magnitude. Wagner et al., Proc Natl Acad Sci U.S.A. 89(17):7934-8 (1992); Midoux et al., Nucl Acids Res. 21(4):871-8 (1993); Kichler et al., Bioconjug Chem 8(2):213-21 (1997); Wagner, Adv Drug Deliv Rev 38(3):279-289 (1999); Zhang et al., J Gene Med 3(6):560-8 (2001). Some artificial peptides such as GALA have been also used as fusogenic peptides. See, for example, Li et al., Adv Drug Deliv Rev 56(7):967-85 (2004) and Sasaki et al., Anal Bioanal Chem 391(8):2717-27 (2008). The fusogenic peptide of the glycoprotein H from herpes simplex virus improves the endosomal release of DNA/Lipofectamine lipoplexes and transgene expression in human cell (Tu and Kim, J Gene Med 10(6):646-54 (2008).

PCT Patent Publication No. WO 2002/044206 describes a class of proteins derived from the family Reoviridae that promote membrane fusion. These proteins are exemplified by the p14 protein from reptilian reovirus and the p16 protein from aquareovirus. PCT Patent Publication No. WO 2012/040825 describes recombinant polypeptides for facilitating membrane fusion, which polypepides have at least 80% sequence identity with the ectodomain of p14 fusion-associated small transmembrane (FAST) protein and having a functional myristoylation motif, a transmembrane domain from a FAST protein and a sequence with at least 80% sequence identity with the endodomain of p15 FAST protein. The '825 PCT further describes the addition of a targeting ligand to the recombinant polypeptide for selective fusion. The recombinant polypeptides presented in the '825 PCT can be incorporated within the membrane of a liposome to facilitate the delivery of nucleic acids. Fusogenix liposomes for delivering therapeutic compounds, including nucleic acids, to the cytoplasm of a mammalian cell, which reduce liposome disruption and consequent systemic dispersion of the cargo nucleic acid and/or uptake into endosomes and resulting nucleic acid destruction are available commercially from Innovascreen Inc. (Halifax, Nova Scotia, Calif.).

2. Nanoparticles

A wide variety of inorganic nanoparticles, including gold, silica, iron oxide, titanium, hydrogels, and calcium phosphates have been described for the delivery of nucleic acids and can be adapted for the delivery of the expression constructs described herein. See, for example Wagner and Bhaduri, Tissue Engineering 18(1):1-14 (2012) (describing inorganic nanoparticles for delivery of nucleic acid sequences); Ding et al., Mol Ther e-pub (2014) (describing gold nanoparticles for nucleic acid delivery); Zhang et al., Langmuir 30(3):839-45 (2014) (describing titanium dioxide nanoparticles for delivery of DNA oligonucleotides); Xie et al., Curr Pharm Biotechnol 14(10):918-25 (2014) (describing biodegradable calcium phosphate nanoparticles fro gene delivery); Sizovs et al., J Am Chem Soc 136(1):234-40 (2014) (describing sub-30 monodisperse oligonucleotide nanoparticles).

Among the advantages of inorganic vectors are their storage stability, low immunogenicity, and resistance to microbial attack. Nanoparticles of less than 100 nm can efficiently trap nucleic acids and allows its escape from endosomes without degradation. Inorganic nanoparticles exhibit improved in vitro transfection for attached cell lines due to their high density and preferential location on the base of the culture dish. Quantum dots have been described that permit the coupling of nucleic acid delivery with stable fluorescence markers.

Hydrogel nanoparticles of defined dimensions and compositions, can be prepared via a particle molding process referred to as PRINT (Particle Replication in Non-wetting Templates), and can be used as delivery vectors for the expression constructs disclosed herein. Nucleic acids can be encapsulated in particles through electrostatic association and physical entrapment. To prevent the disassociation of cargo nucleic acids from nanoparticles following systemic administration, a polymerizable conjugate with a degradable, disulfide linkage can be employed.

The PRINT technique permits the generation of engineered nanoparticles having precisely controlled properties including size, shape, modulus, chemical composition and surface functionality for enhancing the targeting of the expression cassette to a target cell. See, e.g., Wang et al., J Am Chem Soc 132:11306-11313 (2010); Enlow et al., Nano Lett 11:808-813 (2011); Gratton et al., Proc Natl Acad Sci USA 105:11613-11618 (2008); Kelly, J Am Chem Soc 130:5438-5439 (2008); Merkel et al. Proc Natl Acad Sci USA 108:586-591 (2011). PRINT is also amenable to continuous roll-to-roll fabrication techniques that permit the scale-up of particle fabrication under good manufacturing practice (GMP) conditions.

Nanoparticles can be encapsulated with a lipid coating to improveoral bioavailability, minimize enzymatic degradation and cross blood brain barrier. The nanoparticle surface can also be PEGylated to improve water solubility, circulation in vivo, and stealth properties.

3. Viral Vectors

A wide variety of viral vectors are well known by and readily available to those of skill in the art, including, for example, herpes simplex viral vectors lentiviral vectors, adenoviral vectors, and adeno-associated viral vectors, which viral vectors can be adapted for use in the systems disclosed herein for the delivery of nucleic acids, in particular nucleic acids comprising an expression cassette for the target cell specific expression of a therapeutic protein.

The tropisms of natural or engineered viruses towards specific receptors are the foundations for constructing viral vectors for delivery of nucleic acids. The attachment of these vectors to a target cell is contingent upon the recognition of specific receptors on a cell surface by a ligand on the viral vector. Viruses presenting very specific ligands on their surfaces anchor onto the specific receptors on a cell. Viruses can be engineered to display ligands for receptors presented on the surface of a target cell of interest. The interactions between cell receptors and viral ligands are modulated in vivo by toll like receptors.

The entry of a viral vector into a cell, whether via receptor mediated endocytosis or membrane fusion, requires a specific set of domains that permit the escape of the viral vector from endosomal and/or lysosomal pathways. Other domains facilitate entry into nuclei. Replication, assembly, and latency determine the dynamics of interactions between the vector and the cell and are important considerations in the choice of a viral vector, as well as in engineering therapeutic cargo carrying cells, in designing cancer suicide gene therapies.

Herpes simplex virus (HSV) belongs to a family of herpesviridae, which are enveloped DNA viruses. HSV binds to cell receptors through orthologs of their three main ligand glycoproteins: gB, gH, and gL, and sometimes employ accessory proteins. These ligands play decisive roles in the primary routes of virus entry into oral, ocular, and genital forms of the disease. HSV possesses high tropism towards cell receptors of the nervous system, which can be utilized for engineering recombinant viruses for the delivery of expression cassettes to target cells, including senescent cells, cancer cells, and cells infected with an infectious agent. Therapeutic bystander effects are enhanced by inclusion of connexin coding sequences into the constructs. Herpes Simplex Virus vectors for the delivery of nucleic acids to target cells have been reviewed in Anesti and Coffin, Expert Opin Biol Ther 10(1):89-103 (2010); Marconi et al., Adv Exp Med Biol 655:118-44 (2009); and Kasai and Saeki, Curr Gene Ther 6(3):303-14 (2006).

Lentivirus belongs to a family of retroviridae, which are enveloped, single stranded RNA retroviruses and include the Human immunodeficiency virus (HIV). HIV envelope protein binds CD4, which is present on the cells of the human immune system such as CD4+T cells, macrophages, and dendritic cells. Upon entry into a cell, the viral RNA genome is reverse transcribed into double-stranded DNA, which is imported into the cell nucleus and integrated into the cellular DNA. HIV vectors have been used to deliver the therapeutic genes to leukemia cells. Recombinant lentiviruses have been described for mucin-mediated delivery of nucleic acids into pancreatic cancer cells, to epithelial ovarian carcinoma cells, and to glioma cells, without substantial non-specific delivery to normal cells. Lentiviral vectors for the delivery of nucleic acids to target cells have been reviewed in Primo et al., Exp Dermatol 21(3):162-70 (2012); Staunstrup and Mikkelsen, Curr Gene Ther 11(5):350-62 (2011); and Dreyer, Mol Biotechnol 47(2):169-87 (2011).

Adenovirus is a non-enveloped virus consisting of a double-stranded, linear DNA genome and a capsid. Naturally, adenovirus resides in adenoids and may be a cause of upper respiratory tract infections. Adenovirus utilizes a cell's coxsackievirus and adenovirus receptor (CAR) for the adenoviral fiber protein for entry into nasal, tracheal, and pulmonary epithelia. CARs are expressed at low levels on senescent and cancer cells. Recombinant adenovirus can be generated that are capable of nucleic acid deliver to target cells. Replication-competent adenovirus-mediated suicide gene therapy (ReCAP) is in the clinical trials for newly-diagnosed prostate cancer. Adenovirus vectors for the delivery of nucleic acids to target cells have been reviewed in Huang and Kamihira, Biotechnol Adv. 31(2):208-23 (2013); Alemany, Adv Cancer Res 115:93-114 (2012); Kaufmann and Nettelbeck, Trends Mol Med 18(7):365-76 (2012); and Mowa et al., Expert Opin Drug Deliv 7(12):1373-85 (2010).

Adeno-associated virus (AAV) is a small virus that infects humans and some other primate species. AAV is not currently known to cause disease and consequently the virus causes a very mild immune response. Vectors using AAV can infect both dividing and quiescent cells and persist in an extrachromosomal state without integrating into the genome of the host cell. These features make AAV a very attractive candidate for creating viral vectors for use in the systems of the present disclosure. Adeno-associated virus (AAV) vectors for the delivery of nucleic acids to target cells have been reviewed in Li et al., J. Control Release 172(2):589-600 (2013); Hajitou, Adv Genet 69:65-82 (2010); McCarty, Mol Ther 16(10):1648-56 (2008); and Grimm et al., Methods Enzymol 392:381-405 (2005).

4. Polyplexes

Polyplexes are complexes of polymers with DNA. Polyplexes consist of cationic polymers and their fabrication is based on self-assembly by ionic interactions. One important difference between the methods of action of polyplexes and liposomes and lipoplexes is that polyplexes cannot directly release their nucleic acid cargo into the cytoplasm of a target cell. As a result co-transfection with endosome-lytic agents such as inactivated adenovirus is required to facilitate escape from the endocytic vesicle made during particle uptake. better understanding of the mechanisms by which DNA can escape from endolysosomal pathway (i.e., the proton sponge effect) has triggered new polymer synthesis strategies such as the incorporation of protonable residues in polymer backbone and has revitalized research on polycation-based systems. See, e.g., Parhamifar et al., Methods e-pub (2014); Rychgak and Kilbanov, Adv Drug Deliv Rev e-pub (2014); Jafari et al., Curr Med Chem 19(2):197-208 (2012).

Due to their low toxicity, high loading capacity, and ease of fabrication, polycationic nanocarriers exhibit substantial advantages over viral vectors, which show high immunogenicity and potential carcinogenicity and lipid-based vectors which cause dose dependent toxicity. Polyethyleneimine, chitosan, poly(beta-amino esters), and polyphosphoramidate have been described for the delivery of nucleic acids. See, e.g., Buschmann et al., Adv Drug Deliv Rev 65(9):1234-70 (2013). The size, shape, and surface chemistry of these polymeric nano-carriers can be easily manipulated.

5. Dendrimers

Dendrimers are highly branched macromolecules having a spherical shape. The surface of dendrimer particles may be functionalized such as, for example, with positive surface charges (cationic dendrimers), which may be employed for the delivery of nucleic acids. Dendrimer-nucleic acid complexes are taken into a cell via endocytosis. Dendrimers offer robust covalent construction and extreme control over molecule structure and size. Dendrimers are available commercially from Dendritic Nanotechnologies Inc. (Priostar; Mt Pleasant, Mich.), who produce dendrimers using kinetically driven chemistry, which can be adapted fro the delivery of nucleic acids and can transfect cells at a high efficiency with low toxicity.

It will be understood that, while targeted delivey of an expression construct is not required by the systems of the present disclosure and that the targeted reduction, prevention, and/or elimination in the growth and/or survival of a target cell may be achieved by exploiting the intracellular transcriptional machinery of a target cell that is unique to the target cell, it may be desireable, depending upon the precise application contemplated, the incorporate into an otherwise non-specific delivery vector one or more components that facilitate the targeted delivery to a subset of cells at least some of which include a target cell that is susceptible to the growth and/or survival inhibition by the expression constructs of the present disclosure.

The targeted delivery of nucleic acids by liposome, nanoparticle, viral and other vectors described herein has been described in the scientific and patent literature and is well known by and readily available to those of skill in the art. Such targeted delivery technologies may, therefore, be suitably adapted for targeting the delivery of expression constructs of the present disclosure to enhance the specificity of the growth and/or survival reduction, prevention, and/or elimination that is achieved within a target cell. The following examples of targeted delivery technologies are provided herein to exemplify, not to limit, the targeted delivery vectors that may be adapted to achieve the systems of the present disclosure.

Expression Constructs

Expression constructs of the present disclosure comprise: (a) a transcriptional promoter that is responsive to a factor or factors that are produced in a target cell, one or more of which factors is not produced, is produced at a substantially reduced level, is inactive, and/or exhibits a substantially reduced activity in a non-target cell; and (b) a nucleic acid that is operably linked to and under the regulatory control of the transcriptional promoter, wherein the nucleic acid encodes a protein that is capable of reducing, preventing, and/or eliminating the growth and/or survival of a cell in which it is produced, including a target cell.

1. Target Cell Specific Transcriptional Promoters

The present disclosure provides systems comprising a vector for delivering a nucleic acid to a cell wherein the nucleic acid is under the transcriptional control of a promoter that is derepressed or activated in a target cell, but is reprepressed or inactivated in a normal cell, non-target cell.

It will be understood the specificity of the presently disclosed systems toward a target cell is achieved, therefore, through the target cell-specific transcriptional activation of a nucleic acid that encodes a protein that reduces, prevents, and/or eliminates the growth and/or survival of a cell without regard to whether that cell is a target cell. Thus, the target cell specificity of the presently-disclosed systems derives from the transcriptional promoter that regulates the expression of the nucleic acid within the expression cassette in conjunction with transcription-regulatory machinery that is provided by, and unique to, the target cell.

Thus, transcriptional promoters that may be suitably employed in the expression constructs, systems, and methods of the present disclosure include those transcriptional promoters that are capable of promoting the expression of a nucleic acid in a target cell (i.e., a cell that is associated with aging, disease, or other condition), but incapable of, or exhibit a substantially reduced capability of, promoting expression of that nucleic acid in a non-target cell.

Exemplified herein are expression constructs and systems comprising expression constructs wherein the transcriptional promoter is activated in a target cell that is associated with aging, disease, or another condition.

In some embodiments, the present disclosure provides expression constructs and systems that may be employed in methods for the treatment of aging reducing, preventing, and/or eliminating the growth and/or survival of a cell, such as a senescent cell, which is associated with aging. In certain aspects of those embodiments, expression constructs employ a transcriptional promter that is responsive to one or more factors that are produced within a target cell, such as a senescent cell, but are not produced in a non-target cell wherein those one or more factors derepress and/or activate the transcriptional promoter and, as a consequence, promote the expression of a nucleic acid encoding a therapeutic protein that reduces, prevents, and/or eliminates the growth and/or survival of a cell that is associated with aging, including a senescent cell.

The transcriptional promoter itself is the primary mechanism by which senescent cells are preferentially targeted by the systems described in this disclosure. A prototypic example of a target specific transcriptional promoter for use with the systems in this disclosure is a promoter that is only active or mostly active in senescent cells. A number of promoters known by artisans to be active in senescent cells may be used with this system.

In certain aspects of these embodiments wherein the human target cell is a senescent cell, the transcriptional promoter can include the promoter region of p16INK4a/CDKN2A as described in Wang et al., J. Biol. Chem. 276(52):48655-61 (2001), which transcriptional promoter is responsive to activation by a factor such as SP1, ETS1, and ETS2. The transcriptional promoter can also include the promoter region of p21/CDKN1A, which transcriptional promoter is responsive to activation by a factor such as p53/TP53.

In other aspects of these embodiments wherein the human target cell is a cancer cell, such as a brain cancer cell, a prostate cancer cell, a lung cancer cell, a colorectal cancer cell, a breast cancer cell, a liver cancer cell, a hematologic cancer cell, and a bone cancer cell, the transcriptional promoter can include the p21^{cip1/waf1} promoter, the p27^{kip1} promoter, the p57^{kip2} promoter, the TdT promoter, the Rag-1 promoter, the B29 promoter, the Blk promoter, the CD19 promoter, the BLNK promoter, and/or the λ5 promoter, which transcriptional promoter is responsive to activation by one or more transcription factors such as an EBF3, O/E-1, Pax-5, E2A, p53, VP16, MLL, HSF1, NF-IL6, NFAT1, AP-1, AP-2, HOX, E2F3, and/or NF-κB transcription factor, and which transcriptional activation induces the expression of a nucleic acid that encodes a therapeutic protein.

In still further aspects of these embodiments wherein the target cell is a human cell that is infected with an infectious agent, such as a virus, including, for example, a herpes virus, a polio virus, a hepatitis virus, a retrovirus virus, an influenza virus, and a rhino virus, or the target cell is a bacterial cell, the transcriptional promoter can be activated by a factor that is expressed by the infectious agent or bacterial cell, which transcriptional activation induces the expression of a nucleic acid that encodes a therapeutic protein.

The p16 Transcriptional Promoter

In one embodiment, the suicide gene could be placed under control of a p16 promoter, such as a p16Ink4a gene promoter, which is transcriptionally active in senescent, but not in non-senescent cells.

In humans, p16 is encoded by the CDKN2A gene, which gene is frequently mutated or deleted in a wide variety of tumors. p16 is an inhibitor of cyclin dependent kinases such as CDK4 and CDK6, which phosphorylate retinoblastoma protein (pRB) thereby causing the progression from G1 phase to S phase. p16 plays an important role in cell cycle regulation by decelerating cell progression from G1 phase to S phase, and therefore acts as a tumor suppressor that is implicated in the prevention of cancers, including, for example, melanomas, oropharyngeal squamous cell carcinomas, and esophageal cancers. The designation pl6Ink4A refers to the molecular weight (15,845) of the protein encoded by one of the splice variants of the CDKN2A gene and to its role in inhibiting CDK4.

In humans, p16 is encoded by CDKN2A gene, located on chromosome 9 (9p21.3). This gene generates several transcript variants that differ in their first exons. At least three alternatively spliced variants encoding distinct proteins have been reported, two of which encode structurally related isoforms known to function as inhibitors of CDK4. The remaining transcript includes an alternate exon 1 located 20 kb upstream of the remainder of the gene; this transcript contains an alternate open reading frame (ARF) that specifies a protein that is structurally unrelated to the products of the other variants. The ARF product functions as a stabilizer of the tumor suppressor protein p53, as it can interact with and sequester MDM2, a protein responsible for the degradation of p53. In spite of their structural and functional differences, the CDK inhibitor isoforms and the ARF product encoded by this gene, through the regulatory roles of CDK4 and p53 in cell cycle G1 progression, share a common functionality in control of the G1 phase of the cell cycle. This gene is frequently mutated or deleted in a wide variety of tumors and is known to be an important tumor suppressor gene.

Concentrations of p16INK4a increase dramatically as tissue ages. Liu et al., Aging Cell 8(4):439-48 (2009) and Krishnamurthy et al., Nature 443(7110):453-7 (2006). The increased expression of the p16 gene with age reduces the proliferation of stem cells thereby increasing the cellular senescence-associated health risks in a human.

p16 is a cyclin-dependent kinase (CDK) inhibitor that slows down the cell cycle by prohibiting progression from G1 phase to S phase. Normally, CDK4/6 binds cyclin D and forms an active protein complex that phosphorylates retinoblastoma protein (pRB). Once phosphorylated, pRB disassociates from the transcription factor E2F1, liberating E2F1 from its cytoplasm bound state allowing it to enter the nucleus. Once in the nucleus, E2F1 promotes the transcription of target genes that are essential for transition from G1 to S phase.

p16 acts as a tumor suppressor by binding to CDK4/6 and preventing its interaction with cyclin D. This interaction ultimately inhibits the downstream activities of transcription factors, such as E2F1, and arrests cell proliferation. This pathway connects the processes of tumor oncogenesis and senescence, fixing them on opposite ends of a spectrum. On one end, the hypermethylation, mutation, or deletion of p16 leads to downregulation of the gene and can lead to cancer through the dysregulation of cell cycle progression. Conversely, activation of p16 through the ROS pathway, DNA damage, or senescence leads to the build up of p16 in tissues and is implicated in aging of cells.

Regulation of p16 is complex and involves the interaction of several transcription factors, as well as several proteins involved in epigenetic modification through methylation and repression of the promoter region. PRC1 and PRC2 are two protein complexes that modify the expression of p16 through the interaction of various transcription factors that execute methylation patterns that can repress transcription of p16. These pathways are activated in cellular response to reduce senescence.

The p21 Transcriptional Promoter

A nucleic acid encoding a therapeutic protein could be placed under the control of the p21/CDKN1A transcriptional promoter that is often transcriptionally active in senescent, and cancerous or pre-cancerous cells. p53/TP53 plays a central role in the regulation of p21 and, therefore, in the growth arrest of cells when damaged. p21 protein binds directly to cyclin-CDK complexes that drive the cell cycle and inhibits their kinase activity thereby causing cell cycle arrest to allow repair to take place. p21 also mediates growth arrest associated with differentiation and a more permanent growth arrest associated with cellular senescence. The p21 gene contains several p53 response elements that mediate direct binding of the p53 protein, resulting in transcriptional activation of the gene encoding the p21 protein. The role of p53 gene regulation in cellular senescence is described in Kelley et al., Cancer Research 70(9):3566-75. (2010).

2. Nucleic Acids and Therapeutic Proteins Encoded Thereby

Nucleic acids that may be suitably employed in the expression constructs, systems, and methods of the present disclosure encode a protein that is capable of reducing, preventing, and/or eliminating the growth and/or survival of a cell in which it is produced, including a target cell. Thus, the target cell specificity of the presently disclosed expression constructs and systems is achieved by the expression within a target cell, but not within a non-target cell, of a nucleic acid that encodes a therapeutic protein.

Nucleic acids encoding therapeutic proteins that may be employed in the expression constructs and systems of the present disclosure include nucleic acids encoding one or more protein that induces apoptosis in a cell in which it is produced. Exemplified herein are expression constructs and systems comprising one or more “suicide genes,” such as a nucleic acid encoding Herpes Simplex Virus Thymidine Kinase (HSV-TK), cytosine deaminase, CASP3, CASP8, CASP9, BAX, DFF40, cytosine deaminase, or other nucleic acid that encodes a protein that is capable of inducing apoptosis is a cell.

Apoptosis, or programmed cell death (PCD), is a common and evolutionarily conserved property of all metazoans. In many biological processes, apoptosis is required to eliminate supernumerary or dangerous (such as pre-cancerous) cells and to promote normal development. Dysregulation of apoptosis can, therefore, contribute to the development of many major diseases including cancer, autoimmunity and neurodegenerative disorders. In most cases, proteins of the caspase family execute the genetic programme that leads to cell death.

Apoptosis is triggered in a mammalian cell, in particular in a human cell, through the activation of caspase proteins, in particular the caspase proteins CASP3, CASP8, and CASP9. See, for example, Xie et al., Cancer Res 61(18):186-91 (2001); Carlotti et al., Cancer Gene Ther 12(7):627-39 (2005); Lowe et al., Gene Ther 8(18):1363-71 (2001); and Shariat et al., Cancer Res 61 (6):2562-71 (2001).

DNA fragmentation factor (DFF) is a complex of the DNase DFF40 (CAD) and its chaperone/inhibitor DFF45 (ICAD-L). In its inactive form, DFF is a heterodimer composed of a 45 kDa chaperone inhibitor subunit (DFF45 or ICAD), and a 40 kDa latent endonuclease subunit (DFF40 or CAD). Upon caspase-3 cleavage of DFF45, DFF40 forms active endonuclease homo-oligomers. It is activated during apoptosis to induce DNA fragmentation. DNA binding by DFF is mediated by the nuclease subunit, which can also form stable DNA complexes after release from DFF. The nuclease subunit is inhibited in DNA cleavage but not in DNA binding. DFF45 can also be cleaved and inactivated by caspase-7 but not by caspase-6 and caspase-8. The cleaved DFF45 fragments dissociate from DFF40, allowing DFF40 to oligomerise, forming a large complex that cleaves DNA by introducing double strand breaks. Histone H1 confers DNA binding ability to DFF and stimulates the nuclease activity of DFF40. Activation of the apoptotic endonuclease DFF-40 is described in Liu et al., J Biol Chem 274(20):13836-40 (1999).

Thymidine kinase (TK) is an ATP-thymidine 5′-phosphotransferase that is present in all living cells as well as in certain viruses including herpes simplex virus (HSV), varicella zoster virus (VZV), and Epstein-Barr virus (EBV). Thymidine kinase converts deoxythymidine into deoxythymidine 5′-monophosphate (TMP), which is phosphorylated to deoxythymidine diphosphate and to deoxythymidine triphosphate by thymidylate kinase and nucleoside diphosphate kinase, respectively. Deoxythymidine triphosphase is incorporated into cellular DNA by DNA polymerases and viral reverse transcriptases.

When incorporated into DNA, certain dNTP analogs, such as synthetic analogues of 2′-deoxy-guanosine (e.g., Ganciclovir), cause the premature termination of DNA synthesis, which triggers cellular apoptosis.

Within certain embodiments, the expression cassettes and systems of the present disclosure employ a nucleic acid that encodes HSV-TK. Following the administration to a human of a system employing a nucleic acid encoding HSV-TK, an analogue of a 2′-deoxy-nucleotide, such as 2′-deoxy-guanosine, is administered to the human. The HSV-TK efficiently converts the 2′-deoxy-nucleotide analogue into a dNTP analogue, which when incorporated into the DNA induces apoptosis in the target cell.

Cytosine deaminase (CD) catalyzes the hydrolytic conversion in DNA of cytosine to uracil and ammonia. If a CD-modified site is recognized by an endonuclease, the phosphodiester bond is cleaved and, in a normal cell, is repaired by incorporating a new cytosine. In the presence of 5-fluorocytosine (5-FC), cytosine deaminase converts 5-FC into 5-fluorouracil (5-FU), which can inhibit target cell growth. Transgenic expression of CD in a target cell, therefore, reduces the growth and/or survival of the target cell.

The present disclosure provides expression constructs and systems that further comprise one or more safety features to ensure that the expression of a nucleic acid encoding a therapeutic protein is upregulated in appropriate cells, over a desired time period, and/or to a specified level.

Within one such embodiments, expression constructs and systems of the present disclosure employ nucleic acids that encode inducible variants of therapeutic proteins, including, for example, inducible variants of CASP3, CASP8, and CASP9, which require the further contacting of a cell with or administration to a human of a chemical or biological compound that activates the therapeutic protein.

Inducible suicide gene systems are well known and readily available in the art and have been described, for example, in Miller et al., PCT Patent Publication No. WO 2008/154644 and Brenner, US Patent Publication No. 2011/0286980. In addition, Shah et al., Genesis 45(4):104-199 (2007) describe a double-inducible system for Caspase 3 and 9 that employs RU486 and chemical inducers of dimerization (CID). Straathof et al., Blood 105(11):4247-4254 (2005) describe an inducible caspase 9 system in which caspase 9 is fused to a human FK506 binding protein (FKBP) to allow the conditional dimerization using the small molecule AP20187 (ARIAD Pharmaceuticals, Cambridge, Mass.), which is a non-toxic synthetic analog of FK506. Carlotti et al., Cancer Gene Ther 12(7):627-39 (2005) describe an inducible caspase 8 system by employing the ARIAD™ homodimerization system (FKC8; ARIAD Pharmaceuticals).

Full-length inducible caspase 9 (F′F-C-Casp9.I.GFP) comprises a full-length caspase 9, including its caspase recruitment domain (CARD; GenBank NM001 229) linked to two 12 kDa human FK506 binding proteins (FKBP12; GenBank AH002 818) that contain an F36V mutation as described in Clackson et al., Proc. Natl. Acad. Sci. U.S.A. 95:10437-10442 (1998) and are connected by a Ser-Gly-Gly-Gly-Ser linker that connects the FKBPs and caspase 9 to enhance flexibility.

In a further embodiment, the inducible suicide gene could be linked to the nucleic acid sequence for a detectable biomarker such as luciferase or green fluorescent protein to permit the detection of the targeted cells prior to administering a compound to activate an inducible therapeutic protein.

### Compositions and Formulations of Systems Comprising Vectors and Expression Cassettes

The present disclosure provides systems comprising a vector and an expression cassette wherein the expression cassette comprises a transcriptional promoter that is responsive to one or more transcription factors that are expressed in a target cell and a nucleic acid encoding a therapeutic protein. Systems can be administered to a human patient by themselves or in pharmaceutical compositions where they are mixed with suitable carriers or excipient(s) at doses to treat or ameliorate a disease or condition as described herein. Mixtures of these systems can also be administered to the patient as a simple mixture or in pharmaceutical compositions.

Compositions within the scope of this disclosure include compositions wherein the therapeutic agent is a system comprising a vector and an expression cassette in an amount effective to reduce or eliminate the growth and/or survival of a target cell such as a senescent cell, a cancer cell, a cell infected with an infectious agent, a bacterial cell, or a cell that is associated with another disease or condition. Determination of optimal ranges of effective amounts of each component is within the skill of the art. The effective dose is a function of a number of factors, including the specific system, the presence of one or more additional therapeutic agent within the composition or given concurrently with the system, the frequency of treatment, and the patient's clinical status, age, health, and weight.

Compositions comprising a system may be administered parenterally. As used herein, the term “parenteral administration” refers to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion. Alternatively, or concurrently, administration may be orally.

Compositions comprising a system may, for example, be administered intravenously via an intravenous push or bolus. Alternatively, compositions comprising a system may be administered via an intravenous infusion.

Compositions include a therapeutically effective amount of a system, and a pharmaceutically acceptable carrier. As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. Such compositions will contain a therapeutically effective amount of the inhibitor, preferably in purified form, together with a suitable amount of carrier so as to provide the form for proper administration to the patient. The formulation should suit the mode of administration.

Compositions can be formulated in accordance with routine procedures as a pharmaceutical composition adapted for intravenous administration to a human. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The systems disclosed herein can be formulated as neutral or salt forms. Pharmaceutically acceptable salts include those formed with anions such as those derived from hydrochloric, phosphoric, acetic, oxalic, tartaric acids, and the like, and those formed with cations such as those derived from sodium, potassium, ammonium, calcium, ferric hydroxides, isopropylamine, triethylamine, 2-ethylamino ethanol, histidine, procaine, and the like.

### Methods for Treatment of a Disease or Condition Associated with, and for Reducing, Inhibiting, and/or Preventing the Growth and/or Survival of a Cell that is Associated with Aging, Cancer, Infectious Disease, Bacterial Infection, and/or other Disease or Condition

The present disclosure provides methods for reducing, inhibiting, and/or preventing the growth and or survival of a cell that is associated with aging, cancer, infectious disease, bacterial infection, and/or other disease or condition, which methods comprise contacting a target cell or a population of cell comprising a target cell with a system as described herein, which system comprises a vector and an expression construct, which expression construct comprises a transcriptional promoter and a nucleic acid.

The present disclosure also provides methods for the treatment of aging, cancer, infectious disease, bacterial infection, and/or other disease or condition in a patient, which methods comprise the administration of a system as described herein, which system comprises a vector and an expression construct, which expression construct comprises a transcriptional promoter and a nucleic acid.

The present therapeutic methods involve contacting a target cell with, or administering to a human patient, a composition comprising one or more system comprising a vector and an expression cassette to a human patient for reducing and/or eliminating the growth and/or survival of a cell that is associated with senescence, cancer, an infectious disease, a bacterial infection or another disease or condition.

The amount of the system that will be effective in the treatment, inhibition, and/or prevention of aging, cancer, infectious disease, bacterial infection, or other disease or condition that is associated with the elevated expression of one or more transcription factors can be determined by standard clinical techniques. In vitro assays may optionally be employed to help identify optimal dosage ranges. The precise dose to be employed in the formulation will also depend on the route of administration, and the seriousness of the disease or disorder. Effective doses may be extrapolated from dose-response curves derived from in vitro or animal model test systems.

The systems or pharmaceutical compositions of the present disclosure can be tested in vitro, and then in vivo for the desired therapeutic or prophylactic activity, prior to use in humans. For example, in vitro assays to demonstrate the therapeutic or prophylactic utility of a compound or pharmaceutical composition include the effect of a system on a cell line or a patient tissue sample. The effect of the system or pharmaceutical composition thereof on the cell line and/or tissue sample can be determined utilizing techniques known to those of skill in the art including, but not limited to proliferation and apoptosis assays. In accordance with the present disclosure, in vitro assays that can be used to determine whether administration of a specific compound is indicated, include in vitro cell culture assays in which a patient tissue sample is grown in culture, and exposed to or otherwise administered a compound, and the effect of such compound upon the tissue sample is observed.

The present disclosure provides methods for the treatment and growth and/or survival inhibition by administration to a subject of an effective amount of a system or pharmaceutical composition thereof as described herein. In one aspect, the system is substantially purified such that it is substantially free from substances that limit its effect or produce undesired side-effects.

Methods of administration include, but are not limited to, intradermal, intramuscular, intraperitoneal, intravenous, subcutaneous, intranasal, epidural, and oral routes. The systems or compositions thereof may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the inhibitors or compositions into the central nervous system by any suitable route, including intraventricular and intrathecal injection. Intraventricular injection may be facilitated by an intraventricular catheter, for example, attached to a reservoir, such as an Ommaya reservoir. Pulmonary administration can also be employed, for example, by use of an inhaler or nebulizer, and formulation with an aerosolizing agent.

It may be desirable to administer the systems or compositions thereof locally to the area in need of treatment; this may be achieved by, for example, local infusion during surgery, topical application, by injection, by means of a catheter, by means of a suppository, or by means of an implant, said implant being of a porous, non-porous, or gelatinous material, including membranes, such as sialastic membranes, or fibers.

The system can be delivered in a controlled release system placed in proximity of the therapeutic target, thus requiring only a fraction of the systemic dose (see, e.g., Goodson, in Medical Applications of Controlled Release 2:115-138 (1984)).

Intravenous infusion of a compositions comprising a system may be continuous for a duration of at least about one day, or at least about three days, or at least about seven days, or at least about 14 days, or at least about 21 days, or at least about 28 days, or at least about 42 days, or at least about 56 days, or at least about 84 days, or at least about 112 days.

Continuous intravenous infusion of a composition comprising a system may be for a specified duration, followed by a rest period of another duration. For example, a continuous infusion duration may be from about 1 day, to about 7 days, to about 14 days, to about 21 days, to about 28 days, to about 42 days, to about 56 days, to about 84 days, or to about 112 days. The continuous infusion may then be followed by a rest period of from about 1 day, to about 2 days to about 3 days, to about 7 days, to about 14 days, or to about 28 days. Continuous infusion may then be repeated, as above, and followed by another rest period.

Regardless of the precise infusion protocol adopted, it will be understood that continuous infusion of a composition comprising a system will continue until either desired efficacy is achieved or an unacceptable level of toxicity becomes evident.

It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

While various embodiments have been disclosed herein, other embodiments will be apparent to those skilled in the art. The various embodiments disclosed herein are for purposes of illustration and are not intended to be limiting, with the true scope and spirit being indicated by the claims.

The present disclosure will be further described with reference to the following non-limiting examples. The teaching of all patents, patent applications and all other publications cited herein are incorporated by reference in their entirety.

What is claimed is:

1. An expression construct for targeted production of a therapeutic protein within a target cell, said expression construct comprising:
a. a transcriptional promoter that is activated in response to one or more factors each of which is produced within a target cell; and
b. a nucleic acid that is operably linked to and under regulatory control of said transcriptional promoter, wherein said nucleic acid encodes a therapeutic protein that can reduce, prevent, and/or eliminate the growth and/or survival of a cell, including said target cell.

2. The expression construct of claim 1 wherein said transcriptional promoter is activated in said target cell but is not activated in a normal mammalian cell that is not associated with said disease.

3. The expression construct of claim 2 wherein at least one of said factors is not produced in said normal mammalian cell that is not associated with said disease.

4. The expression construct of claim 3 wherein said normal mammalian cell is a normal human cell.

5. The expression construct of claim 4 wherein said normal human cell is selected from the group consisting of a normal skeletal myoblast, a normal adipose cell, a normal cell of the eye, a normal brain cell, a normal liver cell, a normal colon cell, a normal lung cell, a normal pancreas cell, and/or a normal heart cell, which normal cell is not associated with disease, condition, or aging.

6. The expression construct of claim 1 wherein said target cell is selected from the group consisting of a mammalian cell and a bacterial cell.

7. The expression construct of claim 6 wherein said target cell is a mammalian cell.

8. The expression construct of claim 7 wherein said mammalian target cell is a human cell selected from the group consisting of a senescent cell, a cancer cell, and a cell that is infected with an infectious disease agent.

9. The expression construct of claim 8 wherein said human target cell is a senescent cell.

10. The expression construct of claim 9 wherein said transcriptional promoter is selected from the group consisting of a p16INK4a/CDKN2A transcriptional promoter and a p21/CDKN1A transcriptional promoter.

11. The expression construct of claim 9 wherein said transcriptional promoter is responsive to a factor selected from the group consisting of SP1, ETS1, ETS2, and p53/TP53.

12. The expression construct of claim 9 wherein said nucleic acid encodes a therapeutic protein selected from the group consisting of CASP3, CASP8, CASP9, BAX, DFF40, HSV-TK, and cytosine deaminase.

13. The expression construct of claim 9 wherein said therapeutic protein induces cell death in said target cell.

14. The expression construct of claim 13 wherein said induced cell death occurs via a cellular process selected from the group consisting of apoptosis, necrosis/necroptosis, autophagic cell death, endoplasmic reticulum-stress associated cytotoxicity, mitotic catastrophe, paraptosis, pyroptosis, pyronecrosis, and entosifs.

15. The expression construct of claim 8 wherein said human mammalian target cell is a cancer cell.

16. The expression construct of claim 13 wherein said cancer cell is selected from the group consisting of a brain cancer cell, a prostate cancer cell, a lung cancer cell, a colorectal cancer cell, a breast cancer cell, a liver cancer cell, a hematologic cancer cell, and a bone cancer cell.

17. The expression construct of claim 13 wherein said transcriptional promoter is selected from the group consisting of the p21^{cip1/wag1} promoter, the p27^{kip1} promoter, the p57^{kip2} promoter, the TdT promoter, the Rag-1 promoter, the B29 promoter, the Blk promoter, the CD19 promoter, the BLNK promoter, and the λ5 promoter.

18. The expression construct of claim 13 wherein said transcriptional promoter is responsive to a factor selected from the group consisting of an EBF3, O/E-1, Pax-5, E2A, p53, VP16, MLL, HSF1, NF-IL6, NFAT1, AP-1, AP-2, HOX, E2F3, and/or NF-κB transcription factor.

19. The expression construct of claim 13 wherein said nucleic acid encodes a therapeutic protein selected from the group consisting of CASP3, CASP8, CASP9, BAX, DFF40, HSV-TK, and cytosine deaminase.

20. The expression construct of claim 8 wherein said target cell is a human cell that is infected with an infectious disease agent or a bacterial cell.

21. The expression construct of claim 20 wherein said infectious agent is a virus selected from the group consisting of a herpes virus, a polio virus, a hepatitis virus, a retrovirus virus, an influenza virus, and a rhino virus.

22. The expression construct of claim 20 wherein said nucleic acid encodes a therapeutic protein selected from the group consisting of CASP3, CASP8, CASP9, BAX, DFF40, HSV-TK, and cytosine deaminase.

23. A system for the targeted production of a therapeutic protein within a target cell, said system comprising:
a. a vector that is capable of delivering a nucleic acid to a cell, said vector comprising an expression construct; and
b. an expression construct for the targeted production of a therapeutic protein within a target cell, said expression construct comprising:
i. a transcriptional promoter that is activated in response to one or more factors each of which is produced within said target cell; and
ii. a nucleic acid that is operably linked to and under regulatory control of said transcriptional promoter, wherein said nucleic acid encodes a therapeutic protein that can reduce, prevent, and/or eliminate the growth and/or survival of a cell, including said target cell.

24. The system of claim 23 wherein said vector is selected from the group consisting of a liposome, a viral vector, a nanoparticle, a polyplex, and a dendrimer.

25. The system of claim 23 wherein said vector is a liposome wherein said liposome comprises a fusogenic peptide.

26. The system of claim 23 wherein said vector is a viral vector wherein said viral vector comprises is selected from the group consisting of a herpes simplex viral vector, a lentiviral vector, an adenoviral vector, and an adeno-associated viral vector.

27. The system of claim 23 wherein said vector is a nanoparticle wherein said nanoparticle is selected from the group consisting of a including a gold nanoparticle, a silica nanoparticle, an iron oxide nanoparticle, a titanium nanoparticle, a hydrogel nanoparticle, and a calcium phosphate nanoparticle.

28. The system of claim 23 wherein said transcriptional promoter is activated in said target cell but is not activated in a normal mammalian cell that is not associated with said disease.

29. The system of claim 23 wherein at least one of said factors is not produced in said normal mammalian cell that is not associated with said disease.

30. The system of claim 23 wherein said mammalian target cell is a human cell selected from the group consisting of a senescent cell, a cancer cell, and a cell that is infected with an infectious disease agent.

31. The system of claim 30 wherein said human target cell is a senescent cell.

32. The system of claim 30 wherein said transcriptional promoter is selected from the group consisting of a p16INK4a/CDKN2A transcriptional promoter and a p21/CDKN1A transcriptional promoter.

33. The system of claim 32 wherein said transcriptional promoter is responsive to a factor selected from the group consisting of SP1, ETS1, ETS2, and p53/TP53.

34. The system of claim 30 wherein said nucleic acid encodes a therapeutic protein selected from the group consisting of CASP3, CASP8, CASP9, BAX, DFF40, HSV-TK, and cytosine deaminase.

35. The system of claim 34 wherein said therapeutic protein induces cell death in said target cell.

36. The system of claim 35 wherein said induced cell death occurs via a cellular process selected from the group consisting of apoptosis, necrosis/necroptosis, autophagic cell death, endoplasmic reticulum-stress associated cytotoxicity, mitotic catastrophe, paraptosis, pyroptosis, pyronecrosis, and entosifs.

37. The system of claim 30 wherein said human mammalian target cell is a cancer cell.

38. The system of claim 37 wherein said cancer cell is selected from the group consisting of a brain cancer cell, a prostate cancer cell, a lung cancer cell, a colorectal cancer cell, a breast cancer cell, a liver cancer cell, a hematologic cancer cell, and a bone cancer cell.

39. The system of claim 37 wherein said transcriptional promoter is selected from the group consisting of the p21^{cip1/waf1} promoter, the p27^{kip1} promoter, the p57^{kip2} promoter, the TdT promoter, the Rag-1 promoter, the B29 promoter, the Blk promoter, the CD19 promoter, the BLNK promoter, and the λ5 promoter.

40. The system of claim 37 wherein said transcriptional promoter is responsive to a factor selected from the group consisting of EBF3, O/E-1, Pax-5, E2A, p53, VP16, MLL, HSF1, NF-IL6, NFAT1, and NF-κB.

41. The system of claim 37 wherein said nucleic acid encodes a therapeutic protein selected from the group consisting of CASP3, CASP8, CASP9, BAX, DFF40, HSV-TK, and cytosine deaminase.

42. The system of claim 30 wherein said target cell is a human cell that is infected with an infectious disease agent.

43. The system of claim 42 wherein said infectious agent is a virus selected from the group consisting of a herpes virus, a polio virus, a hepatitis virus, a retrovirus virus, an influenza virus, and a rhino virus.

44. The system of claim 42 wherein said nucleic acid encodes a therapeutic protein selected from the group consisting of CASP3, CASP8, CASP9, BAX, DFF40, HSV-TK, and cytosine deaminase.

45. A method for reducing, preventing, and/or eliminating the growth of a target cell, said method comprising:
contacting a target cell with a system for the targeted production of a therapeutic protein within said target cell, said system comprising:
a. a vector that is capable of delivering a nucleic acid to a cell, said vector comprising an expression construct; and
b. an expression construct for the targeted production of a therapeutic protein within a target cell, said expression construct comprising:
i. a transcriptional promoter that is activated in response to one or more factors each of which is produced within said target cell; and
ii. a nucleic acid that is operably linked to and under regulatory control of said transcriptional promoter, wherein said nucleic acid encodes a therapeutic protein,
wherein production of said therapeutic protein in said target cell reduces, prevents, and/or eliminates the growth and/or survival of said target cell.

46. A method for the treatment of a disease or condition in a patient having a target cell, said method comprising:
administering to said patient a system for the targeted production of a therapeutic protein within a target cell, said system comprising:
a. a vector that is capable of delivering a nucleic acid to a cell, said vector comprising an expression construct; and
b. an expression construct for the targeted production of a therapeutic protein within a target cell, said expression construct comprising:
i. a transcriptional promoter that is activated in response to one or more factors each of which is produced within said target cell; and
ii. a nucleic acid that is operably linked to and under regulatory control of said transcriptional promoter, wherein said nucleic acid encodes a therapeutic protein,
wherein production of said therapeutic protein in said target cell reduces, prevents, and/or eliminates the growth and/or survival of said target cell thereby slowing, reversing, and/or eliminating said disease or condition in said patient.
